SEQUENCE LISTING (18 sequences)

SEQ ID NO: 1 - DNA, 24 bp, Artificial Sequence
  Description of Artificial Sequence Synthetic oligonucleotide
ttagggttagggttagggttaggg

SEQ ID NO: 2 - DNA, 17 bp, Artificial Sequence
  Description of Artificial Sequence Synthetic primer
cccagaggtcccagctc

SEQ ID NO: 3 - DNA, 16 bp, Artificial Sequence
  Description of Artificial Sequence Synthetic primer
gagtcacccccgcctt

SEQ ID NO: 4 - DNA, 18 bp, Artificial Sequence
  Description of Artificial Sequence Synthetic primer
tcctctgatcggctgtgg

SEQ ID NO: 5 - DNA, 17 bp, Artificial Sequence
  Description of Artificial Sequence Synthetic primer
agtctgggagcgagcct

SEQ ID NO: 6 - DNA, 19 bp, Artificial Sequence
  Description of Artificial Sequence Synthetic primer
ggagctccacactgtacct

SEQ ID NO: 7 - DNA, 18 bp, Artificial Sequence
  Description of Artificial Sequence Synthetic primer
cctggaaaggacgggcag

SEQ ID NO: 8 - DNA, 15 bp, Artificial Sequence
  Description of Artificial Sequence Synthetic primer
ggaaagcgcgcagcg

SEQ ID NO: 9 - DNA, 17 bp, Artificial Sequence
  Description of Artificial Sequence Synthetic primer
cacagcagcagcagcag

SEQ ID NO: 10 - DNA, 16 bp, Artificial Sequence
  Description of Artificial Sequence Synthetic probe
acacccccttaagagc

SEQ ID NO: 11 - DNA, 14 bp, Artificial Sequence
  Description of Artificial Sequence Synthetic probe
accccgttaagagc

SEQ ID NO: 12 - DNA, 15 bp, Artificial Sequence
  Description of Artificial Sequence Synthetic probe
tgctgctgtcctccg

SEQ ID NO: 13 - DNA, 15 bp, Artificial Sequence
  Description of Artificial Sequence Synthetic probe
tgctgctgacctccg

SEQ ID NO: 14 - DNA, 16 bp, Artificial Sequence
  Description of Artificial Sequence Synthetic probe
caggacagcctgggca

SEQ ID NO: 15 - DNA, 16 bp, Artificial Sequence
  Description of Artificial Sequence Synthetic probe
caggacaccctgggca

SEQ ID NO: 16 - DNA, 12 bp, Artificial Sequence
  Description of Artificial Sequence Synthetic probe
ctgcccccgctg

SEQ ID NO: 17 - DNA, 15 bp, Artificial Sequence
  Description of Artificial Sequence Synthetic probe
ctgctgtccccgctg

SEQ ID NO: 18 - DNA, 15 bp, Artificial Sequence
  Description of Artificial Sequence Synthetic probe
ctgctgcccccgctg

(19) United States
(12) Patent Application Publication
(10) Pub. No.: US 2021/0180131 A1
(43) Pub. Date: Jun. 17, 2021

(54) METHODS FOR DETECTING AND TREATING ATOPIC DERMATITIS

(71) Applicant: AVELLINO LAB USA, INC., Menlo Park, CA (US)

(72) Inventor: Gene Lee, Millbrae, CA (US)

(21) Appl. No.: 16/950,476

(22) Filed: Nov. 17, 2020

Related U.S. Application Data
(63) Continuation of application No. PCT/US2019/032798, filed on May 17, 2019.
(60) Provisional application No. 62/673,068, filed on May 17, 2018.

(51) Int. Cl. C12Q 1/6883; A61N 5/06

(52) U.S. Cl. CPC C12Q 1/6883; A61N 5/0616; A61N 2005/0661; C12Q 2600/106; C12Q 2600/156

(57) ABSTRACT

Methods for detecting single nucleotide polymorphisms (SNPs) associated with atopic dermatitis in a sample from a subject and methods for treating atopic dermatitis in a subject indeed thereof are described.

### FIELD OF THE APPLICATION

This application generally relates to methods for the isolation and detection of disease-associated genetic alleles. In particular, this application relates to methods for the detection of an alleles associated with atopic dermatitis diagnosis and prognosis. This application also generally relates to pharmaceutical compositions and methods for treating atopic dermatitis in a subject in need thereof.

### SEQUENCE LISTING SUBMISSION VIA EFS-WEB

A computer readable text file, entitled “SequenceListing.txt,” created on or about Mar. 8, 2021 with a file size of about 5 KB contains the sequence listing for this application and is hereby incorporated by reference in its entirety.

### BACKGROUND

The present disclosure relates to methods for prognosis and diagnosis of atopic dermatitis by detection of mutated alleles associated with atopic dermatitis. The present disclosure also relates to pharmaceutical compositions and methods for treating atopic dermatitis in a subject in need thereof.

### SUMMARY

The present disclosure provides improved methods for the detection of one or more alleles associated with atopic dermatitis.

The disclosure relates to methods for diagnosing or prognosing atopic dermatitis in a subject, comprising detecting two or more single nucleotide polymorphism (SNPs) in a sample from a subject, wherein the two or more SNPs are selected from the group consisting of rs139653501, rs3812954, rs548525119, rs145018661, rs149117087, rs201486858, rs76655666, rs2229817, rs116914994, rs117501524, rs183149417, rs78272919, and the SNPs listed in Table 2, and wherein the presence of two or more SNPs is indicative of a diagnosis or prognosis of atopic dermatitis in the subject.

In some embodiments, the two or more SNPs comprise rs139653501 and rs3812954; rs139653501 and rs548525119; rs139653501 and rs145018661; rs3812954 and rs548525119; rs3812954 and rs145018661; and/or rs548525119, rs145018661, and the SNPs listed in Table 2. In some embodiments, the two or more SNPs excludes rs139653501, rs3812954, rs548525119, and rs145018661. In additional embodiments, the SNP detection is by a sequencing method. In further embodiments, the subject is Asian. The subject may be Korean, Japanese and/or Chinese. In some embodiments, the method may comprise amplifying a nucleotide molecule from the sample from the subject. In additional embodiments, the detecting comprises detecting the two or more SNPs in a nucleotide molecule from the sample from the subject or its amplicons.

The disclosure also relates to methods for predicting risk of developing atopic dermatitis in a subject, comprising detecting two or more single nucleotide polymorphism (SNPs) in a sample from a subject, wherein the two or more SNPs are selected from the group consisting of rs139653501, rs3812954, rs548525119, rs145018661, rs149117087, rs201486858, rs76655666, rs2229817, rs116914994, rs117501524, rs183149417, rs78272919, and the SNPs listed in Table 2, and wherein the presence of two or more SNPs is indicative of an increased risk of atopic dermatitis in the subject compared to a control subject having the two or more SNPs. In some embodiments, the two or more SNPs comprise rs139653501 and rs3812954; rs139653501 and rs548525119; rs139653501 and rs145018661; rs3812954 and rs548525119; rs3812954 and rs145018661; and/or rs548525119 and rs145018661. In some embodiments, the two or more SNPs excludes rs139653501, rs3812954, rs548525119, and rs145018661. In some embodiments, the two or more SNPs include one, two, three, four, five, six, seven or eight SNPs selected from the group consisting of rs149117087, rs201486858, rs76655666, rs2229817, rs116914994, rs117501524, rs183149417, and rs78272919. In some embodiments, the two or more SNPs include the SNPs listed in Table 2.

In additional embodiments, the SNP detection is by a sequencing method. In further embodiments, the subject is Asian. The subject may be Korean, Japanese and/or Chinese. In some embodiments, the method may comprise amplifying a nucleotide molecule from the sample from the subject. In additional embodiments, the detecting comprises detecting the two or more SNPs in a nucleotide molecule from the sample from the subject or its amplicons.

The disclosure also relates to methods for developing a treatment regimen for the treatment of atopic dermatitis in a subject, comprising detecting two or more single nucleotide polymorphism (SNPs) in a sample from a subject, wherein the two or more SNPs are selected from the group consisting of rs139653501, rs3812954, rs548525119, rs145018661, rs149117087, rs201486858, rs76655666, rs2229817, rs116914994, rs117501524, rs183149417, rs78272919, and the SNPs listed in Table 2, and wherein the presence of two or more SNPs is indicative of the need for an atopic dermatitis treatment regimen in the subject. In some embodiments, the two or more SNPs comprise rs139653501 and rs3812954; rs139653501 and rs548525119; rs139653501 and rs145018661; rs3812954 and rs548525119; rs3812954 and rs145018661; and/or rs548525119 and rs145018661. In some embodiments, the two or more SNPs excludes rs139653501, rs3812954, rs548525119, and rs145018661. In some embodiments, the two or more SNPs include one, two, three, four, five, six, seven or eight SNPs selected from the group consisting of rs149117087, rs201486858, rs76655666, rs2229817, rs116914994, rs117501524, rs183149417, and rs78272919. In some embodiments, the two or more SNPs include the SNPs listed in Table 2. In additional embodiments, the SNP detection is by a sequencing method. In further embodiments, the subject is Asian. The subject may be Korean, Japanese and/or Chinese. In some embodiments, the method may comprise amplifying a nucleotide molecule from the sample from the subject. In additional embodiments, the detecting comprises detecting the two or more SNPs in a nucleotide molecule from the sample from the subject or its amplicons.

The disclosure also relates to methods for treating atopic dermatitis in a subject, the method comprising detecting two or more single nucleotide polymorphism (SNPs) in a sample from a subject, wherein the two or more SNPs are selected from the group consisting of rs139653501, rs3812954, rs548525119, rs145018661, rs149117087, rs201486858, rs76655666, rs2229817, rs116914994, rs117501524, rs183149417, rs78272919, and the SNPs listed in Table 2, and treating atopic dermatitis in the subject. In some embodiments, the two or more SNPs comprise rs139653501 and rs3812954; rs139653501 and rs548525119; rs139653501 and rs145018661; rs3812954 and rs548525119; rs3812954 and rs145018661; and/or rs548525119 and rs145018661. In some embodiments, the two or more SNPs excludes rs139653501, rs3812954, rs548525119, and rs145018661. In some embodiments, the two or more SNPs include one, two, three, four, five, six, seven or eight SNPs selected from the group consisting of rs149117087, rs201486858, rs76655666, rs2229817, rs116914994, rs117501524, rs183149417, and rs78272919. In some embodiments, the two or more SNPs include the SNPs listed in Table 2. In additional embodiments, the SNP detection is by a sequencing method. In further embodiments, the subject is Asian. The subject may be Korean, Japanese and/or Chinese. In some embodiments, the method may comprise amplifying a nucleotide molecule from the sample from the subject. In additional embodiments, the detecting comprises detecting the two or more SNPs in a nucleotide molecule from the sample from the subject or its amplicons.

The present disclosure also provides improved pharmaceutical composition and methods for treating atopic dermatitis.

The disclosure relates to a method for treating atopic dermatitis in a subject in need in thereof, the method comprising treating atopic dermatitis in a subject having two or more single nucleotide polymorphism (SNPs) selected from the group consisting of rs139653501, rs3812954, rs548525119, rs145018661, rs149117087, rs201486858, rs76655666, rs2229817, rs116914994, rs117501524, rs183149417, rs78272919, and the SNPs listed in Table 2. In some embodiments, the two or more SNPs comprise rs139653501 and rs3812954; rs139653501 and rs548525119; rs139653501 and rs145018661; rs3812954 and rs548525119; rs3812954 and rs145018661; and/or rs548525119 and rs145018661. In some embodiments, the two or more SNPs excludes rs139653501, rs3812954, rs548525119, and rs145018661. In some embodiments, the two or more SNPs include one, two, three, four, five, six, seven or eight SNPs selected from the group consisting of rs149117087, rs201486858, rs76655666, rs2229817, rs116914994, rs117501524, rs183149417, and rs78272919. In some embodiments, the two or more SNPs include the SNPs listed in Table 2.

In some embodiments, the method comprises detecting the two or more SNPs in a sample from a subject prior to the treating. The SNP detection may be by a sequencing method, and/or the method may further comprise amplifying a nucleotide molecule from the sample from the subject. In additional embodiments, the detecting comprises detecting the two or more SNPs in a nucleotide molecule from the sample from the subject or its amplicons. In some embodiments, the method may comprise amplifying a nucleotide molecule from the sample from the subject. In additional embodiments, the detecting comprises detecting the two or more SNPs in a nucleotide molecule from the sample from the subject or its amplicons.

In some embodiments, the treatment comprises topically applying moisturizer, corticosteriod, steroids, anti-histamines, or antibiotics to rash on the subject; exposing ultraviolet (UV) light to rash on the subject; or administering steroids, anti-histamines, antibiotics, cyclosporine or interferon to the subject.

In some embodiments, the treatment comprises administering to the subject one or more wild type peptides or proteins corresponding to one or more mutant type peptides or proteins resulted from the two or more SNPs described herein.

In some embodiments, the treatment comprises (i) replacing one or more mutant type sequences of the two or more SNPs to one or more corresponding wild type sequences, (ii) inactivating the one or more mutant type sequences, or (iii) administering the one or more corresponding wild type sequences to the subject.

In some embodiments, the treatment comprises using Zinc Finger Nuclease (ZFN), Transcription Activator-Like Effector Nuclease (TALEN), CRISPR/CAS nuclease system or RNA interference (RNAi).

In further embodiments, the subject is AfroAmerican, Caucasian, Hispanic, or Asian. The subject may be Korean, Japanese and/or Chinese.

### DETAILED DESCRIPTION

The detection of disease-related SNPs is an increasingly more important tool for the diagnosis and prognosis of various medical conditions. With regard to atopic dermatitis, the present disclosure provides methods for detection of mutant alleles and use of this information in or to diagnose a subject with atopic dermatitis as well as to predict the risk of an individual in developing atopic dermatitis. The present disclosure also provides methods for treating atopic dermatitis in a subject in need thereof, in particular in a subject having 2, 3, 4, 5, 6, 7, 8, 9, 10, 11, 12, 13, 14, 15, 16, 17, 18, 19, 20, 21, 22, 23, 24, 25, 26, 27, 28, 29 or 30 single nucleotide polymorphism (SNPs) selected from the group consisting of rs139653501, rs3812954, rs548525119, rs145018661, rs149117087, rs201486858, rs76655666, rs2229817, rs116914994, rs117501524, rs183149417, and rs78272919, and the SNPs listed in Table 2.

The term “invention” or “present invention” as used herein is not meant to be limiting to any one specific embodiment of the invention but applies generally to any and all embodiments of the invention as described in the claims and specification.

As used herein, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure. It should be understood that the use of “and/or” is defined inclusively such that the term “a, b and/or c” should be read to include the sets of “a,” “b,” “c,” “a and b,” “b and c,” “c and a,” and “a, b and c.”

As used herein, the term “about” means modifying, for example, lengths of nucleotide sequences, degrees of errors, dimensions, the quantity of an ingredient in a composition, concentrations, volumes, process temperature, process time, yields, flow rates, pressures, and like values, and ranges thereof, refers to variation in the numerical quantity that may occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of, for example, a composition, formulation, or cell culture with a particular initial concentration or mixture, and amounts that differ due to mixing or processing a composition or formulation with a particular initial concentration or mixture. Whether modified by the term “about” the claims appended hereto include equivalents to these quantities. The term “about” further may refer to a range of values that are similar to the stated reference value. In certain embodiments, the term “about” refers to a range of values that fall within 50, 25, 10, 9, 8, 7, 6, 5, 4, 3, 2, 1 percent or less of the stated reference value.

In one aspect, the disclosure relates to a method for treating atopic dermatitis in a subject in need in thereof, the method comprising treating atopic dermatitis in a subject having 2, 3, 4, 5, 6, 7, 8, 9, 10, 11 or 12 single nucleotide polymorphism (SNPs) selected from the group consisting of rs139653501, rs3812954, rs548525119, rs145018661, rs149117087, rs201486858, rs76655666, rs2229817, rs116914994, rs117501524, rs183149417, and rs78272919. In another aspect, the disclosure relates to a method for treating atopic dermatitis in a subject in need in thereof, the method comprising treating atopic dermatitis in a subject having 2, 3, 4, 5, 6, 7, 8, 9, 10, 11 or 12 single nucleotide polymorphism (SNPs) selected from the group consisting of the SNPs listed in Table 2. In some embodiments, the two or more SNPs comprise rs139653501 and rs3812954; rs139653501 and rs548525119; rs139653501 and rs145018661; rs3812954 and rs548525119; rs3812954 and rs145018661; and/or rs548525119 and rs145018661. In some embodiments, the two or more SNPs excludes rs139653501, rs3812954, rs548525119, and rs145018661. In some embodiments, the two or more SNPs include one, two, three, four, five, six, seven or eight SNPs selected from the group consisting of rs149117087, rs201486858, rs76655666, rs2229817, rs116914994, rs117501524, rs183149417, and rs78272919.

As used herein, the term “polymorphism” and variants thereof refers to the occurrence of two or more alternative genomic sequences or alleles between or among different genomes or individuals. The terms “genetic mutation” or “genetic variation” and variants thereof include polymorphisms.

As used herein the term “single nucleotide polymorphism” (“SNP”) and variants thereof refers to a site of one nucleotide that varies between alleles. A single nucleotide polymorphism (SNP) is a single base change or point mutation but also includes the so-called “indel” mutations (insertions or deletions of a nucleotide), resulting in genetic variation between individuals. SNPs, which make up about 90% of all human genetic variation, occur every 100 to 300 bases along the 3-billion-base human genome. However, SNPs can occur much more frequently in other organisms like viruses. SNPs can occur in coding or non-coding regions of the genome. A SNP in the coding region may or may not change the amino acid sequence of a protein product. A SNP in a non-coding region can alter promoters or processing sites and may affect gene transcription and/or processing. Knowledge of whether an individual has particular SNPs in a genomic region of interest may provide sufficient information to develop diagnostic, preventive and therapeutic applications for a variety of diseases.

In some embodiments, the subject is Asian. The subject may be Korean, Japanese and/or Chinese. In additional embodiments, the subject may be human.

In one aspect, the disclosure relates to a pharmaceutical composition for treating atopic dermatitis. In some embodiments, the method described herein comprises administering any pharmaceutical composition known in the art for treating the atopic dermatitis to a subject. In additional embodiments, the treatment comprises exposing a therapeutically effective amount of ultraviolet (UV) light to rash on the subject.

In additional embodiments, the treatment comprises administering a therapeutically effective amount of the pharmaceutical composition to the subject. The pharmaceutical composition may be applied topically on rash of the subject. The pharmaceutical composition may include moisturizer, corticosteroid, steroids, anti-histamines, antibiotics, cyclosporine, and/or interferon.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the human subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit in a human subject. A prophylactic effect includes delaying or eliminating the appearance of a disease or condition, delaying or eliminating the onset of symptoms of a disease or condition, slowing, halting, or reversing the progression of a disease or condition, or any combination thereof.

In some embodiments, the treatment comprises administering to the subject one or more wild type peptide or proteins corresponding to one or more mutant type peptide or proteins resulted from the two or more SNPs described herein. In additional embodiments, the treatment comprises administering to the subject one or more antibody that binds to the one or more mutant type peptide or proteins.

The term “antibody” refers to a protein molecule functioning as a receptor that specifically recognizes an antigen, and includes an immunoglobulin molecule immunologically reactive with a specific antigen. The term also includes polyclonal antibodies, monoclonal antibodies, whole antibodies and antibody fragments. Further, the term also include chimeric antibodies (for example, humanized murine antibodies), bivalent or bispecific molecules (for example, bispecific antibodies), dibodies, triabodies and tetrabodies. The whole antibodies have two full-length light chains and two full-length heavy chains, and each of the light chains is linked to the heavy chain by a disulfide bond. The whole antibodies include IgA, IgD, IgE, IgM and IgG, and IgG has subtypes, including IgG1, IgG2, IgG3 and IgG4. The antibody fragments refer to fragments having a function of binding to antigens and include Fab, Fab′, F(ab′)2 and Fv. Fab has light chain and heavy chain variable regions, a light chain constant region and a first heavy chain constant region (CH1 domain) and includes one antigen-binding site. Fab′ differs from Fab in that it has a hinge region including at least cysteine residue in the C-terminal region of the heavy chain CH1 domain. F(ab′)₂ antibody is prepared by a disulfide bond between cysteine residues in the hinge region of Fab′. Fv (variable fragment) refers to the minimum antibody fragment having only a heavy chain variable region and a light chain variable region. Double-stranded Fv (dsFv) has a heavy chain variable region linked to a light chain variable region by a disulfide bond, and single-chain Fv (scFv) generally has a heavy chain variable region covalently linked to a light chain variable region by a peptide linker. Such antibody fragments can be obtained using proteases (for example, Fab fragments can be obtained by cleaving whole antibody with papain, and F(ab′)₂ fragments can be obtained by cleaving whole antibody with pepsin). The antibody fragments may be constructed by genetic recombination technology.

In further embodiments, the disclosure also relates to methods of screening for the antibody that binds to the one or more mutant type peptide or proteins. Such antibodies may be screened using the technology known in the art. For example, it is reported that a human IgE monoclonal antibody and a fragment thereof can be screened by using a phage display method (e.g., Steinverger (1996) J. Biol. Chem. 271, 10967-10972). A human IgE monoclonal antibody having a desired bioactivity may be obtained by preparing a human IgE naive (i.e., non-immunized) library using filamentous phages such as M13 that express proteins from total RNA extracted from human IgE producing cells, and screening the library by panning.

In yet further embodiments, the disclosure also relates to methods of manufacturing the antibody that binds to the one or more mutant type peptide or proteins. As an antibody that binds to the mutant type proteins described herein, antibodies derived from human, mouse, rat, rabbit, or goat including polyclonal or monoclonal antibodies, complete or shorten (e.g., F(ab′)₂, Fab′, Fab, or Fv fragment) antibodies, chimeric antibodies, humanized antibodies, or completely humanized antibodies will be acceptable. Such antibodies can be manufactured using the mutant type proteins described herein as an antigen according to well-known production methods of antibody or antiserum. The polyclonal antibodies can be manufactured according to well-known methods. For example, they can be manufactured by separation and refinement of the antibody of which a mixture of an antigen and a carrier protein is immunized to suitable animal, and an antibody inclusion to the antigen is gathered from the immunized animal. As such animal, mouse, rat, sheep, goat, rabbit, and guinea pig are generally enumerated. To improve the antibody producibility, Freund's complete adjuvant or Freund's incomplete adjuvant can be administered with the antigen. The administering is usually executed once every two weeks about 3-10 times in total. The polyclonal antibody can be gathered from the immunized animal's blood and peritoneal fluid, etc. by the above method. The measurement of the polyclonal antibody's titer in antiserum can be measured by ELISA. The separation and refinement of the polyclonal antibody can be executed by refining techniques that use active adsorbents such as antigen binding solid phase, protein A, or protein G, etc., salting-out, alcohol precipitation, isoelectric precipitation, electrophoresis, adsorption and desorption with ion exchanger, ultracentrifugation, or separation and refinement of immunoglobulins such as gel filtration technique, etc. The monoclonal antibody producing cells can be prepared as hybridomas to be possible to subculture which produce the monoclonal antibody by selecting the individual of which the antibody titre is confirmed in an antigen immunized animals, gathering the spleen or the lymph node on day 2-5 after the final immunization, and fusing the antibody producing cells included in them with homogeneous or heterozoic myeloma cells. The antigen itself or with the carrier and the diluent is administered to the part in which the antibody production is possible. To improve the antibody producibility, Freund's complete adjuvant or Freund's incomplete adjuvant can be administered with the antigen. According to the method of calling “DNA immunization”, animals are immunized. This method is a method using a phenomenon in which antigen-expressing vectors are introduced into the part and are taken into myocytes on the process of tissue repair, and expresses the antigenic protein (Nature Immunology (2001), vol. 2, issue 3, p. 261-267) after Cardiotoxin is treated to immune animal's tibialis anterior muscle of hind leg.

In some embodiments, the treatment comprises (i) replacing one or more mutant type sequences of the two or more SNPs to one or more corresponding wild type sequences, (ii) inactivating the one or more mutant type sequences, or (iii) administering the one or more corresponding wild type sequences to the subject. In some embodiments, the treatment comprises a known gene therapy, including, but not limited to, gene editing by Zinc Finger Nuclease (ZFN), Transcription Activator-Like Effector Nuclease (TALEN), CRISPR/CAS nuclease system or RNA interference (RNAi). For example, the treatment comprises TALEN-mediated DNA editing as described in U.S. Patent No. 2016/0367588, which is incorporated herein by reference in its entirety. The treatment may comprise using CRISPR/CAS9 nuclease system as described in U.S. Pat. No. 9,512,446, which is incorporated herein by reference in its entirety. In further embodiments, the treatment comprises a known gene therapy, including, but not limited to, vectors comprising the wild-type sequences. For example, vectors described in U.S. Pat. Nos. 7,001,769, 6,261,834, 5,252,479, and 5,670,488, all of which are incorporated by reference herein in their entirety.

In some embodiments, the invention provides a pharmaceutical composition for oral administration containing the active pharmaceutical ingredient or combination of active pharmaceutical ingredients, such as the peptide, proteins and/or antibodies described herein, and a pharmaceutical excipient suitable for oral administration.

In some embodiments, the invention provides a solid pharmaceutical composition for oral administration containing: (i) an effective amount of an active pharmaceutical ingredient or combination of active pharmaceutical ingredients, and (ii) a pharmaceutical excipient suitable for oral administration. In selected embodiments, the composition further contains (iii) an effective amount of a third active pharmaceutical ingredient and optionally (iv) an effective amount of a fourth active pharmaceutical ingredient.

In some embodiments, the pharmaceutical composition may be a liquid pharmaceutical composition suitable for oral consumption. Pharmaceutical compositions of the invention suitable for oral administration can be presented as discrete dosage forms, such as capsules, sachets, or tablets, or liquids or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The invention further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Each of the active pharmaceutical ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

In some embodiments, the disclosure also relates to a pharmaceutical composition for injection containing an active pharmaceutical ingredient or combination of active pharmaceutical ingredients described herein, and a pharmaceutical excipient suitable for injection.

The forms in which the compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid and thimerosal.

Sterile injectable solutions are prepared by incorporating an active pharmaceutical ingredient or combination of active pharmaceutical ingredients in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

In some embodiments, the invention provides a kit comprising (1) a composition comprising a therapeutically effective amount of an active pharmaceutical ingredient or combination of active pharmaceutical ingredients or a pharmaceutically acceptable salt, solvate, hydrate, cocrystal, or prodrug thereof, and (2) a diagnostic test for determining whether a patient's atopic dermatitis is a particular subtype of atopic dermatitis. Any of the foregoing diagnostic methods may be utilized in the kit.

The kits described above are for use in the treatment of the diseases and conditions described herein. In an embodiment, the kits are for use in the treatment of atopic dermatitis. In some embodiments, the kits are for use in treating atopic dermatitis.

In an embodiment, the kits of the present invention are for use in the treatment of atopic dermatitis described herein.

The amounts of the pharmaceutical compositions administered using the methods herein will be dependent on the human or mammal being treated, the severity of the disorder or condition, the rate of administration, the disposition of the active pharmaceutical ingredients and the discretion of the prescribing physician. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of the pharmaceutical compositions and active pharmaceutical ingredients may be provided in units of mg/kg of body mass or in mg/m2 of body surface area.

In some embodiments, the invention includes a methods of treating atopic dermatitis in a human subject suffering from the atopic dermatitis in a subject having two or more single nucleotide polymorphism (SNPs) selected from the group consisting of rs139653501, rs3812954, rs548525119, and rs145018661, the method comprising the steps of administering a therapeutically effective dose of an active pharmaceutical ingredient to the human subject.

In some embodiments, a pharmaceutical composition or active pharmaceutical ingredient is administered in a single dose. Such administration may be by injection, e.g., intravenous injection, in order to introduce the active pharmaceutical ingredient quickly. However, other routes, including the oral route, may be used as appropriate. A single dose of a pharmaceutical composition may also be used for treatment of an acute condition.

In some embodiments, a pharmaceutical composition or active pharmaceutical ingredient is administered in multiple doses. In an embodiment, a pharmaceutical composition is administered in multiple doses. Dosing may be once, twice, three times, four times, five times, six times, or more than six times per day. Dosing may be once a month, once every two weeks, once a week, or once every other day. In other embodiments, a pharmaceutical composition is administered about once per day to about 6 times per day. In some embodiments, a pharmaceutical composition is administered once daily, while in other embodiments, a pharmaceutical composition is administered twice daily, and in other embodiments a pharmaceutical composition is administered three times daily.

Administration of the active pharmaceutical ingredients in the methods of the invention may continue as long as necessary. In selected embodiments, a pharmaceutical composition is administered for more than 1, 2, 3, 4, 5, 6, 7, 14, or 28 days. In some embodiments, a pharmaceutical composition is administered for less than 28, 14, 7, 6, 5, 4, 3, 2, or 1 day. In some embodiments, a pharmaceutical composition is administered chronically on an ongoing basis—e.g., for the treatment of chronic effects. In some embodiments, the administration of a pharmaceutical composition continues for less than about 7 days. In yet another embodiment the administration continues for more than about 6, 10, 14, 28 days, two months, six months, or one year. In some cases, continuous dosing is achieved and maintained as long as necessary.

In some embodiments, an effective dosage of an active pharmaceutical ingredient disclosed herein is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of an active pharmaceutical ingredient disclosed herein is about 25 mg, about 50 mg, about 75 mg, about 100 mg, about 125 mg, about 150 mg, about 175 mg, about 200 mg, about 225 mg, or about 250 mg.

In some embodiments, an effective dosage of an active pharmaceutical ingredient disclosed herein is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg. In some embodiments, an effective dosage of an active pharmaceutical ingredient disclosed herein is about 0.35 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg.

In some embodiments, an effective dosage of an active pharmaceutical ingredient disclosed herein is in the range of about 1 mg to about 500 mg, about 10 mg to about 300 mg, about 20 mg to about 250 mg, about 25 mg to about 200 mg, about 1 mg to about 50 mg, about 5 mg to about 45 mg, about 10 mg to about 40 mg, about 15 mg to about 35 mg, about 20 mg to about 30 mg, about 23 mg to about 28 mg, about 50 mg to about 150 mg, about 60 mg to about 140 mg, about 70 mg to about 130 mg, about 80 mg to about 120 mg, about 90 mg to about 110 mg, or about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 207 mg. In some embodiments, an effective dosage of an active pharmaceutical ingredient disclosed herein is about 25 mg, about 50 mg, about 75 mg, about 100 mg, about 125 mg, about 150 mg, about 175 mg, about 200 mg, about 225 mg, or about 250 mg.

In some embodiments, an active pharmaceutical ingredient is administered at a dosage of 10 to 200 mg BID, including 50, 60, 70, 80, 90, 100, 150, or 200 mg BID. In some embodiments, an active pharmaceutical ingredient is administered at a dosage of 10 to 500 mg BID, including 1, 5, 10, 15, 25, 50, 75, 100, 150, 200, 300, 400, or 500 mg BID.

In some instances, dosage levels below the lower limit of the aforesaid ranges may be more than adequate, while in other cases still larger doses may be employed without causing any harmful side effect—e.g., by dividing such larger doses into several small doses for administration throughout the day.

An effective amount of the combination of the active pharmaceutical ingredient may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

In some embodiments, the method of treating atopic dermatitis described herein comprises detecting the two or more SNPs in a sample from the subject prior to the treating. The SNP detection may be by a sequencing method, and/or the method may further comprise amplifying a nucleotide molecule from the sample from the subject. In additional embodiments, the detecting comprises detecting the two or more SNPs in a nucleotide molecule from the sample from the subject or its amplicons. In some embodiments, the method may comprise amplifying a nucleotide molecule from the sample from the subject. In additional embodiments, the detecting comprises detecting the two or more SNPs in a nucleotide molecule from the sample from the subject or its amplicons.

The term “primer” and variants thereof refers to an oligonucleotide that acts as a point of initiation of DNA synthesis in a polymerase chain reaction (PCR). A primer is usually about 10 to about 35 nucleotides in length and hybridizes to a region complementary to the target sequence.

The term “probe” and variants thereof (e.g., detection probe) refers to an oligonucleotide that hybridizes to a target nucleic acid in a PCR reaction. Target sequence refers to a region of nucleic acid that is to be analyzed and comprises the polymorphic site of interest.

The hybridization occurs in such a manner that the probes within a probe set may be modified to form a new, larger molecular entity (e.g., a probe product). The probes herein may hybridize to the nucleic acid regions of interest under stringent conditions. As used herein the term “stringency” is used in reference to the conditions of temperature, ionic strength, and the presence of other compounds such as organic solvents, under which nucleic acid hybridizations are conducted. “Stringency” typically occurs in a range from about Tm° C. to about 20° C. to 25° C. below Tm. A stringent hybridization may be used to isolate and detect identical polynucleotide sequences or to isolate and detect similar or related polynucleotide sequences. Under “stringent conditions” the nucleotide sequence, in its entirety or portions thereof, will hybridize to its exact complement and closely related sequences. Low stringency conditions comprise conditions equivalent to binding or hybridization at 68° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5×Denhardt's reagent (50×Denhardt's contains per 500 ml: 5 g Ficoll (Type 400), 5 g BSA) and 100 μg/ml denatured salmon sperm DNA followed by washing in a solution comprising 2.0+SSPE, 0.1% SDS at room temperature when a probe of about 100 to about 1000 nucleotides in length is employed. It is well known in the art that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol), as well as components of the hybridization solution may be varied to generate conditions of low stringency hybridization different from, but equivalent to, the above listed conditions. In addition, conditions which promote hybridization under conditions of high stringency (e.g., increasing the temperature of the hybridization and/or wash steps, the use of formamide in the hybridization solution, etc.) are well known in the art. High stringency conditions, when used in reference to nucleic acid hybridization, comprise conditions equivalent to binding or hybridization at 68° C. in a solution consisting of 5+SSPE, 1% SDS, 5×Denhardt's reagent and 100 μg/ml denatured salmon sperm DNA followed by washing in a solution comprising 0.1+SSPE and 0.1% SDS at 68° C. when a probe of about 100 to about 1000 nucleotides in length is employed.

Unless defined otherwise, all technical and scientific terms used herein have the same meaning as commonly understood by those of ordinary skill in the art to which the invention pertains. Although any methods and materials similar or equivalent to those described herein can be used in the practice or testing of the present invention, various embodiments of methods and materials are specifically described herein.

In general, the work conducted thus far primarily makes use of micro-satellite genotyping and micro-chip technologies (SNP arrays) to interrogate regions of interest within the genome. In comparison, our study utilized Next Gen Sequencing (NGS) technology to identify and to validate genetic variants that contribute to the etiology of the disease. The study involved a whole exome sequencing approach (ACE Platform™; Personalis Inc., Menlo Park, Calif.) in which the ˜22,000 genes that comprise the human exome were captured and sequenced; single point mutations or variants were identified.

In one aspect, the disclosure provides methods for isolating genomic samples to identify and validate single nucleotide polymorphism detection. In some embodiments, the genomic samples may be selected from the group consisting of isolated cells, whole blood, serum, plasma, urine, saliva, sweat, fecal matter, and tears.

In some embodiments, the genomic sample is plasma or serum, and the method further comprises isolating the plasma or serum from a blood sample of the subject.

In some embodiments, the method includes providing a sample of cells from a subject. In some embodiments, the cells are collected by contacting a cellular surface of a subject with a substrate capable of reversibly immobilizing the cells onto a substrate.

The disclosed methods are applicable to a variety of cell types obtained from a variety of samples. In some embodiments, the cell type for use with the disclosed methods include but is not limited to epithelial cells, endothelial cells, connective tissue cells, skeletal muscle cells, endocrine cells, cardiac cells, urinary cells, melanocytes, keratinocytes, blood cells, white blood cells, buffy coat, hair cells (including, e.g., hair root cells) and/or salival cells. In some embodiments, the cells are epithelial cells. In some embodiments, the cells are subcapsular-perivascular (epithelial type 1); pale (epithelial type 2); intermediate (epithelial type 3); dark (epithelial type 4); undifferentiated (epithelial type 5); and large-medullary (epithelial type 6). In some embodiments, the cells are buccal epithelial cells (e.g., epithelial cells collected using a buccal swap). In some embodiments, the sample of cells used in the disclosed methods include any combination of the above identified cell types.

In some embodiments, the method includes providing a sample of cells from a subject. In some embodiments, the cells provided are buccal epithelial cells.

The cell sample is collected by any of a variety of methods which allow for reversible binding of the subjects cells to the substrate. In some embodiments, the substrate is employed in a physical interaction with the sample containing the subject's cells in order to reversibly bind the cells to the substrate. In some embodiments, the substrate is employed in a physical interaction with the body of the subject directly in order to reversibly bind the cells to the substrate. In some embodiments, the sample is a buccal cell sample and the sample of buccal cells is collected by contacting a buccal membrane of the subject (e.g., the inside of their cheek) with a substrate capable of reversibly immobilizing cells that are dislodged from the membrane. In such embodiments, the swab is rubbed against the inside of the subject's cheek with a force equivalent to brushing a person's teeth (e.g., a light amount of force or pressure). Any method which would allow the subject's cells to be reversibly bound to the substrate is contemplated for use with the disclosed methods.

In some embodiments, the sample is advantageously collected in a non-invasive manner. As such sample collection is accomplished anywhere and by almost anyone. For example, in some embodiments, the sample is collected at a physician's office, at a subject's home, or at a facility where a medical procedure is performed or to be performed. In some embodiments the subject, the subject's doctor, nurses or a physician's assistant or other clinical personnel collects the sample.

In some embodiments the substrate is made of any of a variety of materials to which cells are reversibly bound. Exemplary substrates include those made of rayon, cotton, silica, an elastomer, a shellac, amber, a natural or synthetic rubber, cellulose, BAKELITE, NYLON, a polystyrene, a polyethylene, a polypropylene, a polyacrylonitrile, or other materials or combinations thereof. In some embodiments, the substrate is a swab having a rayon tip or a cotton tip.

In some embodiments, the substrate containing the sample is freeze-thawed one or more times (e.g., after being frozen, the substrate containing the sample is thawed, used according to the present methods and re-frozen) and or used in the present methods.

In another aspect, a variety of lysis solutions have been described and are known to those of skill in the art. Any of these well-known lysis solutions can be employed with the present methods in order to isolate nucleic acids from a sample. Exemplary lysis solutions include those commercially available, such as those sold by INVITROGEN®, QIAGEN®, LIFE TECHNOLOGIES® and other manufacturers, as well as those which can be generated by one of skill in a laboratory setting. Lysis buffers have also been well described and a variety of lysis buffers can find use with the disclosed methods, including for example those described in Molecular Cloning (three volume set, Cold Spring Harbor Laboratory Press, 2012) and Current Protocols (Genetics and Genomics; Molecular Biology; 2003-2013), both of which are incorporated herein by reference for all purposes.

Cell lysis is a commonly practiced method for the recovery of nucleic acids from within cells. In many cases, the cells are contacted with a lysis solution, commonly an alkaline solution comprising a detergent, or a solution of a lysis enzyme. Such lysis solutions typically contain salts, detergents and buffering agents, as well as other agents that one of skill would understand to use. After full and/or partial lysis, the nucleic acids are recovered from the lysis solution.

In some embodiments, cells are resuspended in an aqueous buffer, with a pH in the range of from about pH 4 to about 10, about 5 to about 9, about 6 to about 8 or about 7 to about 9.

In some embodiments, the buffer salt concentration is from about 10 mM to about 200 mM, about 10 mM to about 100 mM or about 20 mM to about 80 mM.

In some embodiments, the buffer further comprises chelating agents such as ethylenediaminetetraacetic acid (EDTA) or ethylene glycol tetraacetic acid (EGTA).

In some embodiments, the lysis solution further comprises other compounds to assist with nucleic acid release from cells such as polyols, including for example but not limited to sucrose, as well as sugar alcohols such as maltitol, sorbitol, xylitol, erythritol, and/or isomalt. In some embodiments, polyols are in the range of from about 2% to about 15% w/w, or about 5% to about 15% w/w or about 5% to about 10% w/w.

In some embodiments, the lysis solutions further comprises surfactants, such as for example but not limited to Triton X-100, SDS, CTAB, X-114, CHAPS, DOC, and/or NP-40. In some embodiments such surfactants are in the range of from about 1% to about 5% w/w, about 1% to about 4% w/w, or about 1% to about 3% w/w.

In embodiments, the lysis solution further comprises chaotropes, such as for example but not limited to urea, sodium dodecyl sulfate and/or thiourea. In some embodiments, the chaotrope is used at a concentration in the range of from about 0.5 M to 8 M, about 1 M to about 6 M, about 2 M to about 6 M or about 1 M to 3 M.

In some embodiments, the lysis solution further comprises one or more additional lysis reagents and such lysis reagents are well known in the art. In some embodiments, such lysis reagents include cell wall lytic enzymes, such as for example but not limited to lysozyme. In some embodiments, lysis reagents comprise alkaline detergent solutions, such as 0.1 aqueous sodium hydroxide containing 0.5% sodium dodecyl sulphate.

In some embodiments, the lysis solution further comprises aqueous sugar solutions, such as sucrose solution and chelating agents such as EDTA, for example the STET buffer. In certain embodiments, the lysis reagent is prepared by mixing the cell suspension with an equal volume of lysis solution having twice the desired concentration (for example 0.2 sodium hydroxide, 1.0% sodium dodecyl sulphate).

In some embodiments, after the desired extent of lysis has been achieved, the mixture comprising lysis solution and lysed cells is contacted with a neutralizing or quenching reagent to adjust the conditions such that the lysis reagent does not adversely affect the desired product. In some embodiments, the pH is adjusted to a pH of from about 5 to about 9, about 6 to about 8, about 5 to about 7, about 6 to about 7 or about 6.5 to 7.5 to minimize and/or prevent degradation of the cell contents, including for example but not limited to the nucleic acids. In some embodiments, when the lysis reagent comprises an alkaline solution, the neutralizing reagent comprises an acidic buffer, for example an alkali metal acetate/acetic acid buffer. In some embodiments, lysis conditions, such as temperature and composition of the lysis reagent are chosen such that lysis is substantially completed while minimizing degradation of the desired product, including for example but not limited to nucleic acids.

Any combination of the above can be employed by one of skill, as well as combined with other known and routine methods, and such combinations are contemplated by the present invention.

In another aspect, the nucleic acids, including for example but not limited to genomic DNA, are isolated from lysis buffer prior to performing subsequent analysis. In some embodiments, the nucleic acids are isolated from the lysis buffer prior to the performance of additional analyses, such as for example but not limited to real-time PCR analyses. Any of a variety of methods useful in the isolation of small quantities of nucleic acids are used by various embodiments of the disclosed methods. These include but are not limited to precipitation, gel filtration, density gradients and solid phase binding. Such methods have also been described in for example, Molecular Cloning (three volume set, Cold Spring Harbor Laboratory Press, 2012) and Current Protocols (Genetics and Genomics; Molecular Biology; 2003-2013), incorporated herein by reference for all purposes.

Nucleic Acid precipitation is a well know method for isolation that is known by those of skill in the art. A variety of solid phase binding methods are also known in the art including but not limited to solid phase binding methods that make use of solid phases in the form of beads (e.g., silica, magnetic), columns, membranes or any of a variety other physical forms known in the art. In some embodiments, solid phases used in the disclosed methods reversibly bind nucleic acids. Examples of such solid phases include so-called “mixed-bed” solid phases are mixtures of at least two different solid phases, each of which has a capacity to nucleic acids under different solution conditions, and the ability and/or capacity to release the nucleic acid under different conditions; such as those described in US Patent Application No. 2002/0001812, incorporated by reference herein in its entirety for all purposes. Solid phase affinity for nucleic acids according to the disclosed methods can be through any one of a number of means typically used to bind a solute to a substrate. Examples of such means include but are not limited to, ionic interactions (e.g., anion-exchange chromatography) and hydrophobic interactions (e.g., reversed-phase chromatography), pH differentials and changes, salt differentials and changes (e.g., concentration changes, use of chaotropic salts/agents). Exemplary pH based solid phases include but are not limited to those used in the INVITROGEN ChargeSwitch Normalized Buccal Kit magnetic beads, to which bind nucleic acids at low pH (<6.5) and releases nucleic acids at high pH (>8.5) and mono-amino-N-aminoethyl (MANAE) which binds nucleic acids at a pH of less than 7.5 and release nucleic acids at a pH of greater than 8. Exemplary ion exchange based substrates include but are not limited to DEA-SEPHAROSE™, Q-SEPHAROSE™, and DEAE-SEPHADEX™ from PHARMACIA (Piscataway, N.J.), DOWEX® I from The Dow Chemical Company (Midland, Mich.), AMBERLITE® from Rohm & Haas (Philadelphia, Pa.), DUOLITE® from Duolite International, In. (Cleveland, Ohio), DIALON TI and DIALON TII.

Any individual method is contemplated for use alone or in combination with other methods, and such useful combination are well known and appreciated by those of skill in the art.

In another aspect, the disclosed methods are used to isolate nucleic acids, such as genomic DNA (gDNA) for a variety of nucleic acid analyses, including genomic analyses. In some embodiments, such analysis includes detection of variety of genetic mutations, which include but are not limited to deletions, insertions, transitions and transversions. In some embodiments, the mutation is a single-nucleotide polymorphism (SNP).

A variety of methods for analyzing such isolated nucleic acids, for example but not limited to genomic DNA (gDNA) are known in the art and include nucleic acid sequencing methods (including Next Generation Sequencing methods), PCR methods (including real-time PCR analysis, microarray analysis, hybridization analysis) as well as any other nucleic acid sequence analysis methods that are known in the art, which include a variety of other methods where nucleic acid compositions are analyzed and which are known to those of skill in the art. See, for example, Molecular Cloning (three volume set, Cold Spring Harbor Laboratory Press, 2012) and Current Protocols (Genetics and Genomics; Molecular Biology; 2003-2013).

In one aspect, the SNP described herein may be detected by sequencing. For example, High-throughput or Next Generation Sequencing (NGS) represents an attractive option for detecting mutations within a gene. Distinct from PCR, microarrays, high-resolution melting and mass spectrometry, which all indirectly infer sequence content, NGS directly ascertains the identity of each base and the order in which they fall within a gene. The newest platforms on the market have the capacity to cover an exonic region 10,000 times over, meaning the content of each base position in the sequence is measured thousands of different times. This high level of coverage ensures that the consensus sequence is extremely accurate and enables the detection of rare variants within a heterogeneous sample. For example, in a sample extracted from formalin-fixed, paraffin-embedded (FFPE) tissue, often a mutation of interest is only present at a frequency of 1% with the wild-type allele comprising the remainder. When this sample is sequenced at 10,000× coverage, then even the rare allele, comprising only 1% of the sample, is uniquely measured 100 times over. Thus, NGS provides reliably accurate results with very high sensitivity, making it ideal for clinical diagnostic testing of FFPEs and other mixed samples.

Examples of sequencing techniques, often referred to as Next Generation Sequencing (NGS) techniques include, but are not limited to Massively Parallel Signature Sequencing (MPSS), Polony sequencing, pyrosequencing, Reversible dye-terminator sequencing, SOLiD sequencing, Ion semiconductor sequencing, DNA nanoball sequencing, Helioscope single molecule sequencing, Single molecule real time (SMRT) sequencing, Single molecule real time (RNAP) sequencing, and Nanopore DNA sequencing.

MPSS was a bead-based method that used a complex approach of adapter ligation followed by adapter decoding, reading the sequence in increments of four nucleotides; this method made it susceptible to sequence-specific bias or loss of specific sequences.

Polony sequencing, combined an in vitro paired-tag library with emulsion PCR, an automated microscope, and ligation-based sequencing chemistry to sequence an E. coli genome at an accuracy of >99.9999% and a cost approximately 1/10 that of Sanger sequencing.

A parallelized version of pyrosequencing, the method amplifies DNA inside water droplets in an oil solution (emulsion PCR), with each droplet containing a single DNA template attached to a single primer-coated bead that then forms a clonal colony. The sequencing machine contains many picolitre-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLiD on the other.

A sequencing technology based on reversible dye-terminators. DNA molecules are first attached to primers on a slide and amplified so that local clonal colonies are formed. Four types of reversible terminator bases (RT-bases) are added, and non-incorporated nucleotides are washed away. Unlike pyrosequencing, the DNA can only be extended one nucleotide at a time. A camera takes images of the fluorescently labeled nucleotides, then the dye along with the terminal 3′ blocker is chemically removed from the DNA, allowing the next cycle.

SOLiD technology employs sequencing by ligation. Here, a pool of all possible oligonucleotides of a fixed length are labeled according to the sequenced position.

Oligonucleotides are annealed and ligated; the preferential ligation by DNA ligase for matching sequences results in a signal informative of the nucleotide at that position. Before sequencing, the DNA is amplified by emulsion PCR. The resulting bead, each containing only copies of the same DNA molecule, are deposited on a glass slide. The result is sequences of quantities and lengths comparable to Illumina sequencing.

Ion semiconductor sequencing is based on using standard sequencing chemistry, but with a novel, semiconductor based detection system. This method of sequencing is based on the detection of hydrogen ions that are released during the polymerization of DNA, as opposed to the optical methods used in other sequencing systems. A micro well containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

DNA nanoball sequencing is a type of high throughput sequencing technology used to determine the entire genomic sequence of an organism. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run.

Helicos Biosciences Corporation's single-molecule sequencing uses DNA fragments with added polyA tail adapters, which are attached to the flow cell surface. The next steps involve extension-based sequencing with cyclic washes of the flow cell with fluorescently labeled nucleotides (one nucleotide type at a time, as with the Sanger method). The reads are performed by the Helioscope sequencer.

Single molecule real time (SMRT) sequencing is based on the sequencing by synthesis approach. The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labeled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand.

Single molecule real time sequencing based on RNA polymerase (RNAP), which is attached to a polystyrene bead, with distal end of sequenced DNA is attached to another bead, with both beads being placed in optical traps. RNAP motion during transcription brings the beads in closer and their relative distance changes, which can then be recorded at a single nucleotide resolution. The sequence is deduced based on the four readouts with lowered concentrations of each of the four nucleotide types (similarly to Sangers method).

Nanopore sequencing is based on the readout of electrical signal occurring at nucleotides passing by alpha-hemolysin pores covalently bound with cyclodextrin. The DNA passing through the nanopore changes its ion current. This change is dependent on the shape, size and length of the DNA sequence. Each type of the nucleotide blocks the ion flow through the pore for a different period of time.

VisiGen Biotechnologies uses a specially engineered DNA polymerase. This polymerase acts as a sensor—having incorporated a donor fluorescent dye by its active centre. This donor dye acts by FRET (fluorescent resonant energy transfer), inducing fluorescence of differently labeled nucleotides. This approach allows reads performed at the speed at which polymerase incorporates nucleotides into the sequence (several hundred per second). The nucleotide fluorochrome is released after the incorporation into the DNA strand.

Mass spectrometry may be used to determine mass differences between DNA fragments produced in chain-termination reactions.

Another NGS approach is sequencing by synthesis (SBS) technology which is capable of overcoming the limitations of existing pyrosequencing based NGS platforms.

Such technologies rely on complex enzymatic cascades for read out, are unreliable for the accurate determination of the number of nucleotides in homopolymeric regions and require excessive amounts of time to run individual nucleotides across growing DNA strands. The SBS NGS platform uses a direct sequencing approach to produce a sequencing strategy with very a high precision, rapid pace and low cost.

One exemplary SBS sequencing is initialized by fragmenting of the template DNA into fragments, amplification, annealing of DNA sequencing primers, and, for example, finally affixing as a high-density array of spots onto a glass chip. The array of DNA fragments are sequenced by extending each fragment with modified nucleotides containing cleavable chemical moieties linked to fluorescent dyes capable of discriminating all four possible nucleotides. The array is scanned continuously by a high-resolution electronic camera (Measure) to determine the fluorescent intensity of each base (A, C, G or T) that was newly incorporated into the extended DNA fragment. After the incorporation of each modified base the array is exposed to cleavage chemistry to break off the fluorescent dye and end cap allowing additional bases to be added. The process is then repeated until the fragment is completely sequenced or maximal read length has been achieved.

In another aspect, real-time PCR is used in detecting gene mutations, including for example but not limited to SNPs. In some embodiments, detection of SNPs in specific gene candidates is performed using real-time PCR, based on the use of intramolecular quenching of a fluorescent molecule by use of a tethered quenching moiety. Thus, according to exemplary embodiments, real-time PCR methods also include the use of molecular beacon technology. The molecular beacon technology utilizes hairpin-shaped molecules with an internally-quenched fluorophore whose fluorescence is restored by binding to a DNA target of interest (See, e.g., Kramer, R. et al. Nat. Biotechnol. 14:303-308, 1996). In some embodiments, increased binding of the molecular beacon probe to the accumulating PCR product is used to specifically detect SNPs present in genomic DNA.

For the design of Real-Time PCR assays, several parts are coordinated, including the DNA fragment that is flanked by the two primers and subsequently amplified, often referred to as the amplicon, the two primers and the detection probe or probes to be used.

In some embodiments, a SNP site in a sample from the subject may be amplified by the amplification methods described herein or any other amplification methods known in the art. The nucleic acids in a sample may or may not be amplified prior to contacting the SNP site with a probe described herein, using a universal amplification method (e.g., whole genome amplification and whole genome PCR).

Real-time PCR relies on the visual emission of fluorescent dyes conjugated to short polynucleotides (termed “detection probes”) that associate with genomic alleles in a sequence-specific fashion. Real-time PCR probes differing by a single nucleotide can be differentiated in a real-time PCR assay by the conjugation and detection of probes that fluoresce at different wavelengths. Real-Time PCR finds use in detection applications (diagnostic applications), quantification applications and genotyping applications.

Several related methods for performing real-time PCR are disclosed in the art, including assays that rely on TAQMAN® probes (U.S. Pat. Nos. 5,210,015 and 5,487,972, and Lee et al., Nucleic Acids Res. 21:3761-6, 1993), molecular beacon probes (U.S. Pat. Nos. 5,925,517 and 6,103,476, and Tyagi and Kramer, Nat. Biotechnol. 14:303-8, 1996), self-probing amplicons (scorpions) (U.S. Pat. No. 6,326,145, and Whitcombe et al., Nat. Biotechnol. 17:804-7, 1999), Amplisensor (Chen et al., Appl. Environ. Microbiol. 64:4210-6, 1998), Amplifluor (U.S. Pat. No. 6,117,635, and Nazarenko et al., Nucleic Acids Res. 25:2516-21, 1997, displacement hybridization probes (Li et al., Nucleic Acids Res. 30:E5, 2002), DzyNA-PCR (Todd et al., Clin. Chem. 46:625-30, 2000), fluorescent restriction enzyme detection (Cairns et al., Biochem. Biophys. Res. Commun. 318:684-90, 2004) and adjacent hybridization probes (U.S. Pat. No. 6,174,670 and Wittwer et al., Biotechniques 22:130-1, 134-8, 1997).

One of the many suitable genotyping procedures is the TAQMAN® allelic discrimination assay. In some instances of this assay, an oligonucleotide probe labeled with a fluorescent reporter dye at the 5′ end of the probe and a quencher dye at the 3′ end of the probe is utilized. The proximity of the quencher to the intact probe maintains a low fluorescence for the reporter. During the PCR reaction, the 5′ nuclease activity of DNA polymerase cleaves the probe, and separates the dye and quencher. This results in an increase in fluorescence of the reporter. Accumulation of PCR product is detected directly by monitoring the increase in fluorescence of the reporter dye. The 5′ nuclease activity of DNA polymerase cleaves the probe between the reporter and the quencher only if the probe hybridizes to the target and is amplified during PCR. The probe is designed to straddle a target SNP position and hybridize to the nucleic acid molecule only if a particular SNP allele is present.

Real-time PCR methods include a variety of steps or cycles as part of the methods for amplification. These cycles include denaturing double-stranded nucleic acids, annealing a forward primer, a reverse primer and a detection probe to the target genomic DNA sequence and synthesizing (i.e., replicating) second-strand DNA from the annealed forward primer and the reverse primer. This three step process is referred to herein as a cycle.

In some embodiments, about 10, 15, 20, 25, 30, 35, 40, 45, 50, 55, or 60 cycles are employed. In some embodiments, about 10 to about 60 cycles, about 20 to about 50 or about 30 to about 40 cycles are employed. In some embodiments, 40 cycles are employed.

In some embodiments, the denaturing double-stranded nucleic acids step occurs at a temperature of about 80° C. to 100° C., about 85° C. to about 99° C., about 90° C. to about 95° C. for about 1 second to about 5 seconds, about 2 seconds to about 5 seconds, or about 3 seconds to about 4 seconds. In some embodiments, the denaturing double-stranded nucleic acids step occurs at a temperature of 95° C. for about 3 seconds.

In some embodiments, the annealing a forward primer, a reverse primer and a detection probe to the target genomic DNA sequence step occurs at about 40° C. to about 80° C., about 50° C. to about 70° C., about 55° C. to about 65° C. for about 15 seconds to about 45 seconds, about 20 seconds to about 40 seconds, about 25 seconds to about 35 seconds. In some embodiments, the annealing a forward primer, a reverse primer and a detection probe to the target genomic DNA sequence step occurs at about 60° C. for about 30 seconds.

In some embodiments, the synthesizing (i.e., replicating) second-strand DNA from the annealed forward primer and the reverse primer occurs at about 40° C. to about 80° C., about 50° C. to about 70° C., about 55° C. to about 65° C. for about 15 seconds to about 45 seconds, about 20 seconds to about 40 seconds, about 25 seconds to about 35 seconds. In some embodiments, the annealing a forward primer, a reverse primer and a detection probe to the target genomic DNA sequence step occurs at about 60° C. for about 30 seconds.

In some embodiments, it was found that about 1 μL, about 2 μL, about 3 μL, about 4 μL or about 5 μL of a genomic DNA sample prepared according to the present methods described herein, are combined with only about 0.05 μL, about 0.10 μL about 0.15 μL, about 0.20 μL, about 0.25 μL or about 0.25 μL of a 30×, 35×, 40×, 45×, 50× or 100× real-time PCR assay mix and distilled water to form the PCR master mix. In some embodiments, the PCR master mix has a final volume of about 5 μL, about 6 μL, about 7 μL, about 8 μL, about 9 μL, about 0 μL, about 11 μL, about 12 μL, about 13 μL, about 14 μL, about 15 μL, about 16 μL, about 17 μL, about 18 μL, about 19 μL or about 20 μL or more. In some embodiments, it was found that 2 μL of a genomic DNA sample prepared as described above, are combined with only about 0.15 μL of a 40× real-time PCR assay mix and 2.85 μL of distilled water in order to form the PCR master mix.

While exemplary reactions are described herein, one of skill would understand how to modify the temperatures and times based on the probe design. Moreover, the present methods contemplate any combination of the above times and temperatures.

In some embodiments, primers are tested and designed in a laboratory setting. In some embodiments, primers are designed by computer based in silico methods. Primer sequences are based on the sequence of the amplicon or target nucleic acid sequence that is to be amplified. Shorter amplicons typically replicate more efficiently and lead to more efficient amplification as compared to longer amplicons.

In designing primers, one of skill would understand the need to take into account melting temperature (Tm; the temperature at which half of the primer-target duplex is dissociated and becomes single stranded and is an indication of duplex stability; increased Tm indicates increased stability) based on GC and AT content of the primers being designed as well as secondary structure considerations (increased GC content can lead to increased secondary structure). Tm's can be calculated using a variety of methods known in the art and those of skill would readily understand such various methods for calculating Tm; such methods include for example but are not limited to those available in online tools such as the Tm calculators available on the World Wide Web at promega.com/techserv/tools/biomath/calc11.htm. Primer specificity is defined by its complete sequence in combination with the 3′ end sequence, which is the portion elongated by Taq polymerase. In some embodiments, the 3′ end should have at least 5 to 7 unique nucleotides not found anywhere else in the target sequence, in order to help reduce false-priming and creation of incorrect amplification products. Forward and reverse primers typically bind with similar efficiency to the target. In some instances, tools such as NCBI BLAST (located on the World Wide Web at ncbi.nlm.nih.gov) are employed to performed alignments and assist in primer design.

Those of skill in the art would be well aware of the basics regarding primer design for a target nucleic acid sequence and a variety of reference manuals and texts have extensive teachings on such methods, including for example, Molecular Cloning (three volume set, Cold Spring Harbor Laboratory Press, 2012) and Current Protocols (Genetics and Genomics; Molecular Biology; 2003-2013) and Real-Time PCR in Microbiology: From Diagnostics to Characterization (Ian M. MacKay, Calster Academic Press; 2007); PrimerAnalyser Java tool available on the World Wide Web at primerdigital.com/tools/PrimerAnalyser.html and Kalendar R, et al. (Genomics, 98(2): 137-144 (2011)), all of which are incorporated herein in their entireties for all purposes.

An additional aspect of primer design is primer complexity or linguistic sequence complexity (see, Kalendar R, et al. (Genomics, 98(2): 137-144 (2011)). Primers with greater linguistic sequence complexity (e.g., nucleotide arrangement and composition) are typically more efficient. In some embodiments, the linguistic sequence complexity calculation method is used to search for conserved regions between compared sequences for the detection of low-complexity regions including simple sequence repeats, imperfect direct or inverted repeats, polypurine and polypyrimidine triple-stranded cDNA structures, and four-stranded structures (such as G-quadruplexes). In some embodiments, linguistic complexity (LC) measurements are performed using the alphabet-capacity L-gram method (see, A. Gabrielian, A. Bolshoy, Computer & Chemistry 23:263-274 (1999) and Y. L. Orlov, V. N. Potapov, Complexity: an internet resource for analysis of DNA sequence complexity, Nucleic Acids Res. 32: W628-W633(2004)) along the whole sequence length and calculated as the sum of the observed range (xi) from 1 to L size words in the sequence divided by the sum of the expected (E) value for this sequence length. Some G-rich (and C-rich) nucleic acid sequences fold into four-stranded DNA structures that contain stacks of G-quartets (see, the World Wide Web at quadruplex.org). In some instances, these quadruplexes are formed by the intermolecular association of two or four DNA molecules, dimerization of sequences that contain two G-bases, or by the intermolecular folding of a single strand containing four blocks of guanines (see, P. S. Ho, PNAS, 91:9549-9553 (1994); I. A. Il'icheva, V. L. Florent'ev, Russian Journal of Molecular Biology 26:512-531(1992); D. Sen, W. Gilbert, Methods Enzymol. 211:191-199 (1992); P. A. Rachwal, K. R. Fox, Methods 43:291-301 (2007); S. Burge, G. N. Parkinson, P. Hazel, A. K. Todd, K. Neidle, Nucleic Acids Res. 34:5402-5415 (2006); A. Guédin, J. Gros, P. Alberti, J. Mergny, Nucleic Acids Res. 38:7858-7868 (2010); O. Stegle, L. Payet, J. L. Mergny, D. J. MacKay, J. H. Leon, Bioinformatics 25:i374-i382 (2009); in some instances, these are eliminated from primer design because of their low linguistic complexity, LC=32% for (TTAGGG)₄.

These methods include various bioinformatics tools for pattern analysis in sequences having GC skew, (G−C)/(G+C), AT skew, (A−T)/(A+T), CG−AT skew, (S−W)/(S+W), or purine-pyrimidine (R−Y)/(R+Y) skew regarding CG content and melting temperature and provide tools for determining linguistic sequence complexity profiles. For example the GC skew in a sliding window of n, where n is a positive integer, bases is calculated with a step of one base, according to the formula, (G−C)/(G+C), in which G is the total number of guanines and C is the total number of cytosines for all sequences in the windows (Y. Benita, et al., Nucleic Acids Res. 31:e99 (2003)). Positive GC-skew values indicated an overabundance of G bases, whereas negative GC-skew values represented an overabundance of C bases. Similarly, other skews are calculated in the sequence. Such methods, as well as others, are employed to determine primer complexity in some embodiments.

According to non-limiting example embodiments, real-time PCR is performed using exonuclease primers (TAQMAN® probes). In such embodiments, the primers utilize the 5′ exonuclease activity of thermostable polymerases such as Taq to cleave dual-labeled probes present in the amplification reaction (See, e.g., Wittwer, C. et al. Biotechniques 22:130-138, 1997). While complementary to the PCR product, the primer probes used in this assay are distinct from the PCR primer and are dually-labeled with both a molecule capable of fluorescence and a molecule capable of quenching fluorescence. When the probes are intact, intramolecular quenching of the fluorescent signal within the DNA probe leads to little signal. When the fluorescent molecule is liberated by the exonuclease activity of Taq during amplification, the quenching is greatly reduced leading to increased fluorescent signal. Non-limiting examples of fluorescent probes include the 6-carboxy-fluorescein moiety and the like. Exemplary quenchers include Black Hole Quencher 1 moiety and the like.

A variety of PCR primers can find use with the disclosed methods. Exemplary primers include but are not limited to those described herein. In some embodiments, a primer set for detecting mutation rs139653501 comprising forward primer CCCAGAGGTCCCAGCTC and reverses primer GAGTCACCCCCGCCTT. In some embodiments, a primer set for detecting mutation rs3812954 comprising forward primer TCCTCTGATCGGCTGTGG and reverses primer AGTCTGGGAGCGAGCCT. In some embodiments, a primer set for detecting mutation rs548525119 comprising forward primer GGAGCTCCACACTGTACCT and reverses primer CCTGGAAAGGACGGGCAG. In some embodiments, a primer set for detecting mutation rs145018661 comprising forward primer GGAAAGCGCGCAGCG and reverses primer CACAGCAGCAGCAGCAG.

A variety of detection probes can find use with the disclosed methods and are employed for genotyping and or for quantification. Detection probes commonly employed by those of skill in the art include but are not limited to hydrolysis probes (also known as TAQMAN® probes, 5′ nuclease probes or dual-labeled probes), hybridization probes, and Scorpion primers (which combine primer and detection probe in one molecule).

In additional embodiments, a detection probe for detecting mutation rs139653501 comprises ACA CCC CCT TAA GAG C and/or a detection probe comprising ACC CCG TTA AGA GC. In additional embodiments, a detection probe for detecting mutation rs3812954 comprises TGC TGC TGT CCT CCG and/or a detection probe comprising TGC TGC TGA CCT CCG. In additional embodiments, a detection probe for detecting mutation rs548525119 comprises CAG GAC AGC CTG GGC A and/or a detection probe comprising CAG GAC ACC CTG GGC A. In additional embodiments, a detection probe for detecting mutation rs145018661 comprises CTG CCC CCG CTG and/or a detection probe comprising CTG CTG TCC CCG CTG.

In some embodiments, detection probes contain various modifications. In some embodiments, detection probes include modified nucleic acid residues, such as but not limited to 2′-O-methyl ribonucleotide modifications, phosphorothioate backbone modifications, phosphorodithioate backbone modifications, phosphoramidate backbone modifications, methylphosphonate backbone modifications, 3′ terminal phosphate modifications and/or 3′ alkyl substitutions.

In some embodiments, the detection probe has increased affinity for a target sequence due to modifications. Such detection probes include detection probes with increased length, as well as detection probes containing chemical modifications. Such modifications include but are not limited to 2′-fluoro (2′-deoxy-2′-fluoro-nucleosides) modifications, LNAs (locked nucleic acids), PNAs (peptide nucleic acids), ZNAs (zip nucleic acids), morpholinos, methylphosphonates, phosphoramidates, polycationic conjugates and 2′-pyrene modifications. In some embodiments, the detector probes contains one or more modifications including 2′ fluoro modifications (aka, 2′-Deoxy-2′-fluoro-nucleosides), LNAs (locked nucleic acids), PNAs (peptide nucleic acids), ZNAs (zip nucleic acids), morpholinos, methylphosphonates, phosphoramidates, and/or polycationic conjugates.

In some embodiments, the detection probes contain detectable moieties, such as those described herein as well as any detectable moieties known to those of skill in the art. Such detectable moieties include for example but are not limited to fluorescent labels and chemiluminescent labels. Examples of such detectable moieties can also include members of FRET pairs. In some embodiments, the detection probe contains a detectable entity.

Examples of fluorescent labels include but are not limited to AMCA, DEAC (7-Diethylaminocoumarin-3-carboxylic acid); 7-Hydroxy-4-methylcoumarin-3; 7-Hydroxycoumarin-3; MCA (7-Methoxycoumarin-4-acetic acid); 7-Methoxycoumarin-3; AMF (4′-(Aminomethyl)fluorescein); 5-DTAF (5-(4,6-Dichlorotriazinyl)aminofluorescein); 6-DTAF (6-(4,6-Dichlorotriazinyl)aminofluorescein); 6-FAM (6-Carboxyfluorescein; aka FAM; including TAQMAN® FAM™); TAQMAN VIC®; 5(6)-FAM cadaverine; 5-FAM cadaverine; 5(6)-FAM ethylenediamme; 5-FAM ethylenediamme; 5-FITC (FITC Isomer I; fluorescein-5-isothiocyanate); 5-FITC cadaverin; Fluorescein-5-maleimide; 5-IAF (5-Iodoacetamidofluorescein); 6-JOE (6-Carboxy-4′,5′-dichloro-2′,7′-dimethoxyfluorescein); 5-CR110 (5-Carboxyrhodamine 110); 6-CR110 (6-Carboxyrhodamine 110); 5-CR6G (5-Carboxyrhodamine 6G); 6-CR6G (6-Carboxyrhodamine 6G); 5(6)-Carboxyrhodamine 6G cadaverine; 5(6)-Carboxyrhodamine 6G ethylenediamme; 5-ROX (5-Carboxy-X-rhodamine); 6-ROX (6-Carboxy-X-rhodamine); 5-TAMRA (5-Carboxytetramethylrhodamine); 6-TAMRA (6-Carboxytetramethylrhodamine); 5-TAMRA cadaverine; 6-TAMRA cadaverine; 5-TAMRA ethylenediamme; 6-TAMRA ethylenediamme; 5-TMR C6 maleimide; 6-TMR C6 maleimide; TR C2 maleimide; TR cadaverine; 5-TRITC; G isomer (Tetramethylrhodamine-5-isothiocyanate); 6-TRITC; R isomer (Tetramethylrhodamine-6-isothiocyanate); Dansyl cadaverine (5-Dimethylaminonaphthalene-1-(N-(5-aminopentyl))sulfonamide); EDANS C2 maleimide; fluorescamine; NBD; and pyrromethene and derivatives thereof.

Examples of chemiluminescent labels include but are not limited to those labels used with Southern Blot and Western Blot protocols (see, for e.g., Sambrook and Russell, Molecular Cloning: A Laboratory Manual, (3rd ed.) (2001); incorporated by reference herein in its entirety). Examples include but are not limited to -(2′-spiroadamantane)-4-methoxy-4-(3″-phosphoryloxy)phenyl-1,2-dioxetane (AMPPD); acridinium esters and adamantyl-stabilized 1,2-dioxetanes, and derivatives thereof.

The labeling of probes is known in the art. The labeled probes are used to hybridize within the amplified region during amplification. The probes are modified so as to avoid them from acting as primers for amplification. The detection probe is labeled with two fluorescent dyes, one capable of quenching the fluorescence of the other dye. One dye is attached to the 5′ terminus of the probe and the other is attached to an internal site, so that quenching occurs when the probe is in a non-hybridized state.

Typically, real-time PCR probes consist of a pair of dyes (a reporter dye and an acceptor dye) that are involved in fluorescence resonance energy transfer (FRET), whereby the acceptor dye quenches the emission of the reporter dye. In general, the fluorescence-labeled probes increase the specificity of amplicon quantification.

Real-time PCR that are used in some embodiments of the disclosed methods also include the use of one or more hybridization probes (i.e., detection probes), as determined by those skilled in the art, in view of this disclosure. By way of non-limiting example, such hybridization probes include but are not limited to one or more of those provided in the described methods. Exemplary probes, such as the HEX channel and/or FAM channel probes, are understood by one skilled in the art.

According to example embodiments, detection probes and primers are conveniently selected e.g., using an in silico analysis using primer design software and cross-referencing against the available nucleotide database of genes and genomes deposited at the National Center for Biotechnology Information (NCBI). Some additional guidelines may be used for selection of primers and/or probes in some embodiments. For example, in some embodiments, the primers and probes are selected such that they are close together, but not overlapping. In some embodiments, the primers may have the same (or close Tm) (e.g., between about 58° C. and about 60° C.). In some embodiments, the Tm of the probe is approximately 10° C. higher than that selected for the Tm of the primers. In some embodiments, the length of the probes and primers is selected to be between about 17 and 39 base pairs, etc. These and other guidelines are used in some instances by those skilled in the art in selecting appropriate primers and/or probes.

In some embodiments, the SNP described herein may be detected by melting curve analysis using the detection probes above. For example, the melting curves of short oligonucleotide probes hybridized to a region containing the SNP of interest may be analyzed. Two probes are used in these reactions, each one being complimentary to a particular allele at the SNP in question. Perfectly matched probes are more stable and have a higher melting temperature compared to mismatched probes. Hence, SNP genotypes are inferred according to the characteristic melting curves produced by annealing and melting either matched or mismatched oligonucleotide probes.

In one aspect, the methods described herein may include detecting the two or more SNPs described herein by hybridizing at least one detection probe to a nucleotide molecule from a sample or its amplicons and detecting the at least one detection probe.

In another aspect, diagnostic testing is employed to determine one or more genetic conditions by detection of any of a variety of mutations. In some embodiments, diagnostic testing is used to confirm a diagnosis when a particular condition is suspected based on for example physical manifestations, signs and/or symptoms as well as family history information. In some embodiments, the results of a diagnostic test assist those of skill in the medical arts in determining an appropriate treatment regimen for a given subject and allow for more personalized and more effective treatment regimens. In some embodiments, a treatment regimen include any of a variety of pharmaceutical treatments, surgical treatments, lifestyles changes or a combination thereof as determined by one of skill in the art.

The nucleic acids obtained by the disclosed methods are useful in a variety of diagnostic tests, including tests for detecting mutations such as deletions, insertions, transversions and transitions. In some embodiments, such diagnostics are useful for identifying unaffected individuals who carry one copy of a gene for a disease that requires two copies for the disease to be expressed, identifying unaffected individuals who carry one copy of a gene for a disease in which the information could find use in developing a treatment regimen, preimplantation genetic diagnosis, prenatal diagnostic testing, newborn screening, genealogical DNA test (for genetic genealogy purposes), presymptomatic testing for predicting or diagnosing atopic dermatitis.

In some embodiments, newborns can be screened. In some embodiments, newborn screening includes any genetic screening employed just after birth in order to identify genetic disorders. In some embodiments, newborn screening finds use in the identification of genetic disorders so that a treatment regimen is determined early in life. Such tests include but are not limited to testing infants for phenylketonuria and congenital hypothyroidism.

In some embodiments, carrier testing is employed to identify people who carry a single copy of a gene mutation. In some cases, when present in two copies, the mutation can cause a genetic disorder. In some cases, one copy is sufficient to cause a genetic disorder. In some embodiments, such information is also useful for individual contemplating procreation and assists individuals with making informed decisions as well as assisting those skilled in the medical arts in providing important advice to individual subjects as well as subjects' relatives.

In some embodiments, predictive and/or presymptomatic types of testing are used to detect gene mutations associated with a variety of disorders. In some cases, these tests are helpful to people who have a family member with a genetic disorder, but who may exhibit no features of the disorder at the time of testing. In some embodiments, predictive testing identifies mutations that increase a person's chances of developing disorders with a genetic basis, including for example but not limited to certain types of atopic dermatitis. In some embodiments, presymptomatic testing is useful in determining whether a person will develop a genetic disorder, before any physical signs or symptoms appear. The results of predictive and presymptomatic testing provides information about a person's risk of developing a specific disorder and help with making decisions about an appropriate medical treatment regimen for a subject as well as for a subject's relatives. Predictive testing is also employed, in some embodiments, to detect mutations which are contra-indicated with certain treatment regimens.

In some embodiments, diagnostic testing also includes pharmacogenomics which includes genetic testing that determines the influence of genetic variation on drug response. Information from such pharmacogenomic analyses finds use in determining and developing an appropriate treatment regimen. Those of skill in the medical arts employ information regarding the presence and/or absence of a genetic variation in designing appropriate treatment regimen.

In some embodiments, diseases whose genetic profiles are determined using the methods of the present disclosure include atopic dermatitis.

In some embodiments, the present methods find use in development of personalized medicine treatment regimens by providing the genomic DNA which is used in determining the genetic profile for an individual. In some embodiments, such genetic profile information is employed by those skilled in the art in order determine and/or develop a treatment regimen. In some embodiments, the presence and/or absence of various genetic variations and mutations identified in nucleic acids isolated by the described methods are used by those of skill in the art as part of a personalized medicine treatment regimen or plan. For example, in some embodiments, information obtained using the disclosed methods is compared to databases or other established information in order to determine a diagnosis for a specified disease and or determine a treatment regimen. In some cases, the information regarding the presence or absence of a genetic mutation in a particular subject is compared to a database or other standard source of information in order to make a determination regarding a proposed treatment regimen. In some cases, the presence of a genetic mutation indicates pursuing a particular treatment regimen. In some cases the absence of a genetic mutation indicates not pursuing a particular treatment regimen.

In some embodiments, information regarding the presence and/or absence of a particular genetic mutation is used to determine the treatment efficacy of treatment with the therapeutic entity, as well as to tailor treatment regimens for treatment with therapeutic entity. In some embodiments, information regarding the presence and/or absence of a genetic mutation is employed to determine whether to pursue a treatment regimen. In some embodiments, information regarding the presence and/or absence of a genetic mutation is employed to determine whether to continue a treatment regimen. In some embodiments, the presence and/or absence of a genetic mutation is employed to determine whether to discontinue a treatment regimen. In other embodiments, the presence and/or absence of a genetic mutation is employed to determine whether to modify a treatment regimen. In some embodiments the presence and/or absence of a genetic mutation is used to determine whether to increase or decrease the dosage of a treatment that is being administered as part of a treatment regimen. In other embodiments, the presence and/or absence of a genetic mutation is used to determine whether to change the dosing frequency of a treatment administered as part of a treatment regimen. In some embodiments, the presence and/or absence of a genetic mutation is used to determine whether to change the number of dosages per day, per week, times per day of a treatment. In some embodiments the presence and/or absence of a genetic mutation is used to determine whether to change the dosage amount of a treatment. In some embodiments, the presence and/or absence of a genetic mutation is determined prior to initiating a treatment regimen and/or after a treatment regimen has begun. In some embodiments, the presence and/or absence of a genetic mutation is determined and compared to predetermined standard information regarding the presence or absence of a genetic mutation.

In some embodiments, a composite of the presence and/or absence of more than one genetic mutation is generated using the disclosed methods and such composite includes any collection of information regarding the presence and/or absence of more than one genetic mutation. In some embodiments, the presence or absence of 2 or more, 3 or more, 4 or more, 5 or more, 6 or more, 7 or more, 8 or more, 9 or more, 10 or more, 20 or more, 30 or more or 40 or more genetic mutations is examined and used for generation of a composite. Exemplary information in some embodiments includes nucleic acid or protein information, or a combination of information regarding both nucleic acid and/or protein genetic mutations. Generally, the composite includes information regarding the presence and/or absence of a genetic mutation. In some embodiments, these composites are used for comparison with predetermined standard information in order to pursue, maintain or discontinue a treatment regimen.

In some embodiments, atopic dermatitis is predicted and/or detected for example through detection of 2, 3, 4, 5, 6, 7, 8, 9, 10, 11 or 12 SNPs selected from but not limited rs139653501, rs3812954, rs548525119, rs145018661, rs149117087, rs201486858, rs76655666, rs2229817, rs116914994, rs117501524, rs183149417, and rs78272919. In additional embodiments, atopic dermatitis is predicted and/or detected for example through detection of rs139653501 and rs3812954; rs139653501 and rs548525119; rs139653501 and rs145018661; rs3812954 and rs548525119; rs3812954 and rs145018661; and/or rs548525119 and rs145018661. In some embodiments, the two or more SNPs excludes rs139653501, rs3812954, rs548525119, and rs145018661. In some embodiments, the two or more SNPs include one, two, three, four, five, six, seven or eight SNPs selected from the group consisting of rs149117087, rs201486858, rs76655666, rs2229817, rs116914994, rs117501524, rs183149417, and rs78272919.

The presence of two or more atopic dermatitis associated SNP alleles in a subject is indicative of a diagnosis or prognosis of atopic dermatitis in the subject. In some embodiments, the subject is determined to have atopic dermatitis if the subject has an atopic dermatitis associated SNP allele percentage count at or above over a particular set threshold. As used herein, a “SNP allele count percentage” means the percentage of SNP alleles of interest detected at a particular set of loci in relation to the total number of loci in the set. Therefore, an “atopic dermatitis associated SNP allele count percentage” means the percentage of atopic dermatitis associated SNP alleles detected at a particular set of loci in relation to the total number of loci in the set. Any of the atopic dermatitis associated SNP composites described herein can be used to determine the atopic dermatitis associated SNP allele count percentage. In some embodiments, 2, 3, or 4 SNPs selected from but not limited to rs139653501, rs3812954, rs548525119, and rs145018661 are used to determine whether a subject has atopic dermatitis or is predicted to develop atopic dermatitis.

In some embodiments, an atopic dermatitis associated SNP allele count percentage of 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%. 91%, 92%, 93%, 94%, 95%, 96%, 97%, 98%, 99% or more is indicative of atopic dermatitis in a subject. In some embodiments, an atopic dermatitis associated SNP allele count percentage of 25% or more is indicative that a subject has atopic dermatitis.

In some embodiments, an atopic dermatitis associated SNP allele count percentage of 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%. 91%, 92%, 93%, 94%, 95%, 96%, 97%, 98%, 99% or less is indicative that a subject does not have atopic dermatitis. In some embodiments, an atopic dermatitis associated SNP allele count percentage of less than 25% is indicative that a subject does not have atopic dermatitis.

In some embodiments, an atopic dermatitis associated SNP allele count percentage of 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%. 91%, 92%, 93%, 94%, 95%, 96%, 97%, 98%, 99% or more is indicative that a subject is at risk to develop atopic dermatitis. In some embodiments, an atopic dermatitis associated SNP allele count percentage of 25% or more is indicative that a subject is at risk to develop atopic dermatitis.

In some embodiments, an atopic dermatitis associated SNP allele count percentage of 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%. 91%, 92%, 93%, 94%, 95%, 96%, 97%, 98%, 99% or less is indicative that a subject is not at risk to develop atopic dermatitis. In some embodiments, an atopic dermatitis associated SNP allele count percentage of less than 25% is indicative that a subject is not at risk to develop atopic dermatitis.

In some embodiments, the detection of two or more SNPs is combined with a physical examination in order to diagnose atopic dermatitis or predict the risk of developing atopic dermatitis. Such a physical examination can include and eye examination as well as ancillary tests to assess corneal curvature, astigmatism and thickness. In some embodiments, the best potential vision of the subject is evaluated. Components of the eye exam can include but are not limited to medical history (including, for example, change in eye glass prescription, decreased vision, history of eye rubbing, medical problems, allergies, and/or sleep patterns); assessment of relevant aspects of the subject's mental and physical status; visual acuity with current correction (the power of the present correction recorded) at distance and when appropriate at near and far distances; measurement of best corrected visual acuity with spectacles and/or hard or gas permeable contact lenses (with refraction when indicated); measurement of pinhole visual acuity; external examination (lids, lashes, lacrimal apparatus, orbit); examination of ocular alignment and motility; assessment of pupillary function; measurement of intraocular pressure (IOP); slit-lamp biomicroscopy of the anterior segment; dilated examination (including for example, dilated examination of the lens, macula, peripheral retina, optic nerve, and vitreous); and Keratometry/Computerized Topography/Computerized Tomography/Ultrasound Pachymetry.

In some embodiments, the detection of two or more SNPs is in combination with one or more indications or signs of atopic dermatitis development in order to diagnose atopic dermatitis or predict the risk of developing atopic dermatitis. In some embodiments, the sign is an early signs of atopic dermatitis.

In some embodiments, the detection of two or more SNPs associated with an increased risk of developing atopic dermatitis can be used to assist with determining a treatment regimen for an individual suspected to have atopic dermatitis or predicted to develop atopic dermatitis in the future.

In some embodiments, the detection of two or more SNPs as described herein can be used to begin an appropriate treatment early in an individual suspected to be a risk of developing atopic dermatitis. In some embodiments, the detection of two or more SNPs that predict and increased risk of developing atopic dermatitis can allow for earlier and/or more frequent monitoring of the skin in order to identify disease onset at an early (i.e., identify early disease onset).

In another aspect, the detection of two or more SNPs as described herein can be used to begin early or regular monitoring in an individual suspected to be a risk of developing atopic dermatitis.

In another aspect, the detection of two or more SNPs as described herein can be used to diagnose atopic dermatitis in a subject.

In one aspect, the disclosure provides methods for treating atopic dermatitis in a subject, the method comprising diagnosing or prognosing atopic dermatitis and treating atopic dermatitis in the subject. In some embodiments, the treating may comprises treating rash on the skin of a subject. In further embodiments, the treating may comprise applying topically applying moisturizer, corticosteriod, steroids, anti-histamines, or antibiotics to rash on the subject; exposing ultraviolet (UV) light to rash on the subject; or administering steroids, anti-histamines, antibiotics, cyclosporine or interferon to the subject.

In another aspect, the disclosure provides a diagnostic kit for diagnosing, prognosing and/or treating atopic dermatitis. Any or all of the reagents described above may be packaged into a diagnostic kit. Such kits include any and/or all of the primers, probes, buffers and/or other reagents described herein in any combination. In some embodiments, the kit includes reagents for detection of 1, 2, 3, 4, 5, 6, 7, 8, 9, 10, 11 or 12 SNPs selected from, but not limited to, rs139653501, rs3812954, rs548525119, rs145018661, rs149117087, rs201486858, rs76655666, rs2229817, rs116914994, rs117501524, rs183149417, and rs78272919.

In some embodiments, the reagents in the kit are included as lyophilized powders. In some embodiments, the reagents in the kit are included as lyophilized powders with instructions for reconstitution. In some embodiments, the reagents in the kit are included as liquids. In some embodiments, the reagents are included in plastic and/or glass vials or other appropriate containers. In some embodiments the primers and probes are all contained in individual containers in the kit. In some embodiments, the primers are packaged together in one container, and the probes are packaged together in another container. In some embodiments, the primers and probes are packaged together in a single container.

In some embodiments, the kit further includes control gDNA and/or DNA samples. In some embodiments, the control DNA sample is normal (e.g., from a subject who does not have KC). In some embodiments, the control DNA sample corresponds to the mutation being detected, including any of SNPs selected from the group consisting of rs139653501, rs3812954, rs548525119, and rs145018661. In some embodiments, the control DNA sample corresponds to the mutation being detected, including any of SNPs selected from the group consisting of rs139653501, rs3812954, rs548525119, and rs145018661. In some embodiments, a control DNA sample corresponds to normal and a mutant DNA sample corresponds to any of rs139653501, rs3812954, rs548525119, and rs145018661 are included.

In some embodiments, the concentration of the control DNA sample is 5 ng/μL, 10 ng/μL, 20 ng/μL, 30 ng/μL, 40 ng/μL, 50 ng/μL, 60 ng/μL, 70 ng/μL, 80 ng/μL, 90 ng/μL, 100 ng/μL, 110 ng/μL, 120 ng/μL, 130 ng/μL, 140 ng/μL, 150 ng/μL, 160 ng/μL, 170 ng/μL, 180 ng/μL, 190 ng/μL or 200 ng/μL. In some embodiments, the concentration of the control DNA sample is 50 ng/μL, 100 ng/μL, 150 ng/μL or 200 ng/μL. In some embodiments, the concentration of the control DNA sample is 100 ng/μL. In some embodiments, the control DNA samples have the same concentration. In some embodiments, the control DNA samples have different concentrations.

In some embodiments, the kit can further include buffers, for example, GTXpress TAQMAN® reagent mixture, or any equivalent buffer. In some embodiments, the buffer includes any buffer described herein.

In some embodiments, the kit can further include reagents for use in cloning, such as vectors (including, e.g., M13 vector).

In some embodiments, the kit further includes reagents for use in purification of DNA.

In some embodiments, the kit further includes instructions for using the kit for the detection of corneal dystrophy in a subject. In some embodiments, these instructions include various aspects of the protocols described herein.

### EXAMPLES: TARGET SNPS FOUND IN NGS DATA

Next Gen Sequencing (NGS) technology is utilized to identify and to validate genetic variants that contribute to the etiology of the disease. The study involved a whole exome sequencing approach (ACE Platform™; Personalis Inc., Menlo Park, Calif.) in which ˜22,000 genes that comprise the human exome were captured and sequenced; single point mutations or variants were identified.

This genomic study involved a patient cohort consisting of 59 individual cases and 13 controls. Total of 4 genes in Table 1 below were identified as significant based primarily on rare SNPs detected.

TABLE 1

| Chr | Start | Ref | Alt | Func. | Gene | Exonic Func. | AA change | cytoBand | dbSNP ID |
|---|---|---|---|---|---|---|---|---|---|
| 16 | 88496531 | C | G | exonic | ZNF469 | nonsynonymous SNV | NM_001127464:c.C2653G:p.L885V | 16q24.2 | rs139653501 |
| 16 | 88502208 | A | T | exonic | ZNF469 | nonsynonymous SNV | NM_001127464:c.A8246T:p.D2749V | 16q24.2 | rs3812954 |
| 9 | 137534094 | C | T | exonic | COL5A1 | nonsynonymous SNV | NM_000093:c.C61T:p.P21S | 9q34.3 | rs548525119 |
| 13 | 110828986 | C | G | exonic | COL4A1 | nonsynonymous SNV | NM_001845:c.G2955C:p.Q985H | 13q34 | rs145018661 |

A whole exome sequencing (WES) method (Personalis Inc., Menlo Park, Calif.) was utilized to carry out next generation sequencing (NGS). The following describes the bioinformatics technique utilized for the determination of the various genes and the correlating SNPs found within the whole exomes of the patients' DNA. The numbers were calculated by adding the individual relative risk score for each SNP. The color scale on the left gives the relative risk for these 4 SNPs, and they are all quite low. The “relative risk” score was based on how these 4 SNPs were represented in the sample cohort of the 59 individual cases and 13 controls.

The genomic studies using NGS technology based on a targeted gene panel were repeated with samples from six keratoconus patients having irritated eyes. The common SNPs identified among the patients are listed in Table 2 below. Table 3 lists SNPs that are also found in other keratoconus patients.

TABLE 2

| | | Reference | Sample | | Protein | |
|---|---|---|---|---|---|---|
| Chromosome | Position | Allele | Allele | Gene Symbol | Variant | dbSNP ID |
| 1 | 11199694 | C | A | MTOR | p.D1632Y | NA |
| 1 | 36563777 | G | A | COL8A2 | p.T502M | 117860804 |
| 2 | 189922049 | G | T | COL5A2 | p.D778E | NA |
| 2 | 227875149 | G | T | COL4A4 | p.L1468M | NA |
| 2 | 227886785 | T | A | COL4A4 | p.M1399L | 149117087 |
| 2 | 227907829 | C | T | COL4A4 | p.G1121R | NA |
| 2 | 189916926 | G | A | COL5A2 | p.A914V | 201486858 |
| 3 | 123356997 | C | T | MYLK | p.V1452M | 76655666 |
| 3 | 123367831 | C | T | MYLK | p.E1292K | NA |
| 5 | 140908381 | T | A | DIAPH1 | p.K969M | NA |
| 5 | 121413208 | C | T | LOX | p.R158Q | 1800449 |
| 6 | 75833112 | G | C | COL12A1 | p.P1130A | NA |
| 7 | 20685484 | C | A | ABCB5 | p.Q262K | 2074000 |
| 7 | 22771039 | T | A | IL6 | p.D162E | 13306435 |
| 9 | 137701066 | G | T | COL5A1 | p.G1135V | NA |
| 9 | 137726950 | C | T | COL5A1 | p.T1757M | 2229817 |
| 9 | 137712038 | C | A | LOC101448202; | p.P1508H | NA |
| | | | | COL5A1 | | |
| 9 | 124065224 | G | A | GSN | p.A114T | 2230287 |
| 10 | 31815894 | A | G | ZEB1 | p.E1012G | NA |
| 14 | 74971769 | C | T | LTBP2 | p.R1429Q | 116914994 |
| 14 | 74976452 | C | T | LTBP2 | p.G1088S | 61505039 |
| 15 | 86686981 | T | A | AGBL1 | p.L56H | NA |
| 15 | 86791030 | A | T | AGBL1 | p.R219W | NA |
| 16 | 75513713 | C | T | CHST6 | p.R5H | NA |
| 16 | 88494747 | C | T | ZNF469 | p.A290V | 117501524 |
| 16 | 88495895 | G | T | ZNF469 | p.A673S | NA |
| 16 | 88504595 | G | A | ZNF469 | p.G3545R | 183149417 |
| 16 | 88494747 | C | T | ZNF469 | p.A290V | 117501524 |
| 16 | 88500348 | G | A | ZNF469 | p.R2129K | 13334190 |
| 16 | 88502208 | A | T | ZNF469 | p.D2749V | 3812954 |
| 17 | 39672190 | G | T | KRT15 | p.L325M | 200152929 |
| 17 | 39673366 | C | G | KRT15 | p.A184P | 78272919 |
| 17 | 39766755 | T | C | KRT16 | p.M370V | 201334428 |
| 17 | 5436263 | C | T | NLRP1 | p.V1029M | 2301582 |
| 17 | 5485367 | A | T | NLRP1 | p.L155H | 12150220 |
| 20 | 25059442 | C | T | VSX1 | p.R217H | 6138482 |

TABLE 3

| Chr | Position | Ref | Alt | Gene | AA Change ref Gene | dbSNP ID | MAF (ExAC_ALL) |
|---|---|---|---|---|---|---|---|
| 2 | 227886785 | T | A | COL4A4 | NM_000092:c.A4195T:p.M1399L | rs149117087 | 0.00078 |
| 2 | 189916926 | G | A | COL5A2 | NM_000393:c.C2741T:p.A914V | rs201486858 | 0.0001 |
| 3 | 123356997 | C | T | MYLK | NM_053026:c.G4675A:p.V1559M | rs76655666 | 0.0003 |
| 9 | 137726950 | C | T | COL5A1 | NM_000093:c.C5270T:p.T1757M | rs2229817 | 0.0121 |
| 14 | 74971769 | C | T | LTBP2 | NM_000428:c.G4286A:p.R1429Q | rs116914994 | 0.004 |
| 16 | 88494747 | C | T | ZNF469 | NM_001127464:c.C869T:p.A290V | rs117501524 | 0.0035 |
| 16 | 88504595 | G | A | ZNF469 | NM_001127464:c.G10633A:p.G3545R | rs183149417 | 0.0002 |
| 17 | 39673366 | C | G | KRT15 | NM_002275:c.G550C:p.A184P | rs78272919 | 0.0013 |

In silico prediction of missense variants: To determine pathogenicity, i.e., likelihood of being damaging and/or altering protein function, the level of agreement from 7 in silico tools was used: SIFT, PolyPhen, PolyPhenv2, LRT, MutationTaster, MutationAssesor, and FATHMM scores. Each tool aims to determine the likely impact on the transcribed amino acid sequence and translated protein domain structure due to the missense change, with each having its own take on what's important or not to look at in this regard. Each of these has a score and then a prediction, with the following possibilities:
D, Deleterious
P, Possibly Deleterious
T/N/B/U, Tolerated/Neutral/Benign/Unknown

Conservation scoring for variants: The following were 3 main scoring systems that took conservation into account and these test how well the surrounding site is conserved across mammals and/or vertebrates.

1. GERP++
a. Citation: ncbi.nlm.nih.gov/pubmed/21152010
b. Score ranging from −12.3 to 6.17, with 6.17 being the most conserved.

2. PhyloP
a. Citation: ncbi.nlm.nih.gov/pubmed/19858363
b. Score calculated using info from 40+ genome alignments to determine conservation. There's a score for vertebrates and mammals.
c. The score range differs for each chromosome, but generally it's around −20 to 10.

3. SiPhy
a. Citation: ncbi.nlm.nih.gov/pmc/articles/PMC2687944/b.
B. As above but based on 29 genome alignments (mammals).
c. Produces a log odds ratio, with the higher value indicating higher conservation. From the way they calculate this, around 10 is one of the highest possible values.

Allele frequency annotations: Where possible, each variant's allele frequency in the Exome Aggregation Consortium (ExAC, exac.broadinstitute.org/), which contains data from 60,706 unrelated individuals, and NHLBI-GO Exome Sequencing Project (NHLBI-ESP, evs.gs.washington.edu/EVS/), which contains data from 6,503 individuals, was checked.

The ExAC populations are as follows:
ExAC_ALL, all ExAC populations
ExAC_AFR, African and African-American
ExAC_AMR, Latino
ExAC_EAS, East-Asian
ExAC_FIN, Finnish
ExAC_NFE, Non-Finnish European
ExAC_SAS, South-Asian
ExAC_OTH, Other

The NHLBI-ESP population is comprised of African- and European-Americans.

Rare variant selection: In order to select variants most likely to be damaging and thus related to disease, criteria were developed to filter results as follows.
1. Not present in controls (where possible).
2. Focus on missense, STOP gain/loss, and nonsense SNVs, and frameshift/non-frameshift InDels.
3. Filtering based on minor allele frequency (MAF):
   a. Koreans, ExAC_EAS≤0.05 or NA
   b. Caucasians, ExAC_ALL≤0.01 or NA
   c. Czechs, ExAC_NFE≤0.05 or NA
   d. Hispanics, ExAC_AMR≤0.01 or NA
   e. African-Americans, ExAC_AFR≤0.01 or NA
4. Relation of gene containing the variant to eye function or known to be involved in disease through gene enrichment analysis using the Database for Annotation,
   Visualization and Integrated Discovery (ncbi.nlm.nih.gov/pmc/articles/PMC2375021/).
5. Pathogenicity of each SNV based on consensus from 7 in silico tools.

### Examples: Diagnosis of Atopic Dermatitis

Blood samples from patients diagnosed with atopic dermatitis are analyzed with the following probes. Each probe is labeled with a fluorescence label.

| Mutation to be detected | Probe sequences |
|---|---|
| rs139653501 | ACA CCC CCT TAA GAG C |
| rs139653501 | ACC CCG TTA AGA GC |
| rs3812954 | TGC TGC TGT CCT CCG |
| rs3812954 | TGC TGC TGA CCT CCG |
| rs548525119 | CAG GAC AGC CTG GGC A |
| rs548525119 | CAG GAC ACC CTG GGC A |
| rs145018661 | CTG CTG CCC CCG CTG |
| rs145018661 | CTG CTG TCC CCG CTG |

Tm analysis is performed in the presence of a plasmid sample from the above blood sample and a comparative plasmid sample. PCR and Tm analysis are performed with a PCR reaction solution using a fully automatic SNPs detection apparatus.

Samples show positive results with at least two probes shown above, indicating the presence of the relevant mutations. Some samples show positive results with all eight probes above. Some samples show positive results for at least one probe per mutation from the list above. Some samples also show positive results with at least one probe detecting rs149117087, rs201486858, rs76655666, rs2229817, rs116914994, rs117501524, rs183149417, or rs78272919.

Alternatively, samples are analyzed by sequencing amplified sequences including the mutation sites, for example, by using Illumina's sequencing machine. The results from the sequencing are the same as the Tm analysis results in that the samples exhibit the same positive and negative results with respect to the presence and absence of the relevant mutations.

Once the atopic dermatitis is diagnosed in a patient, the patient is treated. Some patients are treated by topically applying moisturizer, corticosteriod, steroids, anti-histamines, or antibiotics to the patient. Some patients are treated by exposure to ultraviolet (UV) light. Some patients are treated by administering steroids, anti-histamines, antibiotics, cyclosporine or interferon. Some patients are treated by administering a wild type protein(s) corresponding to a mutant type protein(s) resulted from the two or more SNPs. Symptoms of the atopic dermatitis improve after the treatments described herein.

What is claimed:

1. A method for treating atopic dermatitis in a subject, the method comprising detecting two or more single nucleotide polymorphism (SNPs) in a sample from a subject, wherein the two or more SNPs are selected from the group consisting of rs139653501, rs3812954, rs548525119, rs145018661, rs149117087, rs201486858, rs76655666, rs2229817, rs116914994, rs117501524, rs18149417, and rs78272919, and treating atopic dermatitis in the subject.

2. The method according to claim 1, wherein the two or more SNPs comprise rs139653501, rs3812954, rs548525119, and rs145018661.

3. The method according to claim 1, wherein the two or more SNPs excludes rs139653501, rs3812954, rs548525119, and rs145018661.

4. The method according to claim 1, wherein the two or more SNPs comprise two or more SNPs selected from the group consisting of rs149117087, rs201486858, rs76655666, rs2229817, rs116914994, rs117501524, rs18149417, and rs78272919.

5. The method according to claim 1, wherein the two or more SNPs comprise rs149117087, rs201486858, rs76655666, rs2229817, rs116914994, rs117501524, rs18149417, and rs78272919.

6. The method according to claim 1, wherein said SNP detection is by a sequencing method.

7. The method according to claim 1, wherein the subject is Asian.

8. The method according to claim 1, wherein the subject is Korean, Japanese and/or Chinese.

9. The method according to claim 1, further comprising amplifying a nucleotide molecule from the sample from the subject.

10. The method according to claim 1, wherein the detecting comprises detecting the two or more SNPs in a nucleotide molecule from the sample from the subject or its amplicons.

11. A method for treating atopic dermatitis in a subject in need in thereof, the method comprising treating atopic dermatitis in a subject having two or more single nucleotide polymorphism (SNPs) selected from the group consisting of rs139653501, rs3812954, rs548525119, rs145018661, rs149117087, rs201486858, rs76655666, rs2229817, rs116914994, rs117501524, rs18149117, and rs78272919.

12. The method according to claim 11, further comprising detecting the two or more SNPs in the sample from the subject prior to the treating.

13. The method according to claim 12, wherein said SNP detection is by a sequencing method.

14. The method according to claim 12, further comprising amplifying a nucleotide molecule from the sample from the subject.

15. The method according to claim 12, wherein the detecting comprises detecting the two or more SNPs in a nucleotide molecule from the sample from the subject or its amplicons.

16. The method according to claim 11, wherein the treatment comprises topically applying moisturizer, corticosteriod, steroids, anti-histamines, or antibiotics to rash on the subject.

17. The method according to claim 11, wherein the treatment comprises exposing ultraviolet (UV) light to rash on the subject.

18. The method according to claim 11, wherein the treatment comprises administering steroids, anti-histamines, antibiotics, cyclosporine or interferon to the subject.

19. The method according to claim 11, wherein the treatment comprises administering to the subject one or more wild type proteins corresponding to one or more mutant type proteins resulted from the two or more SNPs.

20. The method according to claim 11, wherein the treatment comprises (i) replacing one or more mutant type sequences of the two or more SNPs with one or more corresponding wild type sequences, (ii) inactivating the one or more mutant type sequences, or (iii) administering the one or more corresponding wild type sequences to the subject.
